(19) United States
(12) Patent Application Publication
(10) Pub. No.: US 2003/0235553 A1
(43) Pub. Date: Dec. 25, 2003

(54) Cosmetic compositions containing at least one silicone-polyamide polymer, at least one oil and at least one film-forming agent and methods of using the same

(75) Inventors: Shaoxiang Lu, Plainsboro, NJ (US); Xavier Blin, Paris (FR); Jean Mondet, Aulnay-sous-Bois (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/166,760

(22) Filed: Jun. 12, 2002

(51) Int. Cl. A61K 7/06; A61K 7/11

(52) U.S. Cl. 424/70.122

(57) ABSTRACT

The invention relates to a physiologically acceptable composition, in particular a cosmetic composition, containing at least one liquid fatty phase structured with at least one structuring polymer of the silicone-polyamide type, the polymer being solid at room temperature and soluble in said oil at a temperature of from 25 to 250° C., and at least one film-forming polymer, said oil having an affinity with said structuring polymer and optionally with said film-forming polymer, and the liquid fatty phase, the polymer and the film-forming polymer forming a physiologically acceptable medium. This composition may be in the form of a stick of lipstick which is stable, which does not exude and whose application produces a glossy deposit with good staying power over time.

The present invention relates to a care and/or treatment and/or make-up composition for the skin, including the scalp, and/or for the lips of human beings, and/or for keratinous materials, such as keratinous fibers, containing a liquid fatty phase, structured with a specific polymer.

This composition can be stable over time and may be in the form of a stick of make-up such as lipstick, the application of which can produce a glossy deposit with good staying power or long-wearing properties.

It is common to find a structured, i.e., gelled and/or rigidified, liquid fatty phase in cosmetic or dermatological products; this is especially the case in solid compositions such as deodorants, lip balms, lipsticks, concealer products, eye shadows and cast foundations. This structuring may be obtained with the aid of waxes and/or fillers. Unfortunately, these waxes and fillers may have a tendency to make the composition matte, which may not always be desirable, in particular for a lipstick or an eye shadow. Consumers are always on the lookout for a lipstick in stick form which can deposit a film with good staying power or long wearing properties but which is also increasingly glossy.

The structuring of the liquid fatty phase may make it possible in particular to limit its exudation (or syneresis) from solid compositions, particularly in hot and humid areas and, furthermore, after deposition on the skin or the lips, to limit the migration of this phase into wrinkles and fine lines, a characteristic particularly desirable in a lipstick or eye shadow. The reason for this is that considerable migration of the liquid fatty phase, particularly when it is charged with coloring agents, may lead to an unpleasant appearance around the lips and the eyes, making wrinkles and fine lines particularly prominent Consumers often state this migration as being a major drawback of conventional lipsticks and eye shadows. The term “migration” means movement of the composition beyond its initial site of application.

Gloss of a lipstick or other cosmetic is generally associated with the nature of the liquid fatty phase. Thus, it may be possible to reduce the amount of waxes and/or fillers in the composition in order to increase the gloss of a lipstick, but in that case the migration of the liquid fatty phase may increase. In other words, the amounts of waxes and of fillers required to prepare a stick of suitable hardness that does not exude at room temperature are a restricting factor on the gloss of the deposit.

To overcome at least one of these drawbacks, it has been envisaged replacing all or some of the waxes and/or fillers with polymers for structuring the liquid fatty phase, of the silicone-polyamide type. Unfortunately, the sticks obtained are not mechanically or thermally stable.

Furthermore, make-up compositions should have good staying power or long-wearing properties over time, i.e., little turning of or change in color over time or a gradual or homogeneous change of the deposit over time. The turning of or change in color of the deposit may be due, for lipsticks, to an interaction with saliva and, for foundations and eye shadows, to an interaction with the sweat and sebum secreted by the skin.

Furthermore, the majority of make-up or care compositions, when they are applied to the skin, eyelashes or lips, exhibit the disadvantage of transferring, that is to say of being at least partly deposited and leaving traces on certain substrates with which they may be brought into contact, in particular a glass, a cup, a cigarette, an item of clothing or the skin. This results in mediocre persistence of the applied film, requiring the regular renewal of the application of the composition, in particular a foundation or lipstick composition. In point of fact, it is the wish of users today to beautify their faces, including the lips, and their bodies while spending the least possible time doing so. Furthermore, the appearance of these unacceptable traces, in particular on blouse collars, can dissuade some women from using this type of make up.

The need thus remains for a composition which does not have at least one of the above drawbacks, which has good stability over time, even in hot atmosphere, and which produces a deposit on the skin or the lips that shows good staying power or long-wearing over time and has a glossy appearance. Furthermore, this composition can be easy to manufacture and can give the deposit a sensation of not drying out, both during application and over time.

One subject of the invention is a care and/or make-up and/or treatment composition for the skin and/or the lips of the face and/or for superficial body growths, i.e., keratinous materials, such as nails or keratinous fibers, which makes it possible to overcome at least one of the drawbacks mentioned above.

The inventors have found, surprisingly, that the use of at least one specific structuring polymer combined with at least one film-forming agent makes it possible to obtain a stick whose application to the lips produces a deposit, which can have noteworthy cosmetic properties. In particular, the deposit has good staying power or long-wearing properties over time, and does not transfer on the support applied on the deposit.

Furthermore, the composition can be stable over time at room temperature (25° C.) as well as high temperature (typically 47° C.). The term “stable” refers to a composition, in particular a stick that is hard and does not collapse over time at room temperature (25° C.) and at 47° C. for at least 1 month.

The invention applies not only to make-up products for the lips, such as lipsticks, lip glosses and lip pencils, but also to care and/or treatment products for the skin, including the scalp, and for the lips, such as antisun care products for the human face, the body or the lips, such as in stick form, make-up removing products for the skin of the face and body, make-up products for the skin, both of the human face and body, such as foundations optionally cast in stick or dish form, concealer products, blushers, eyeshadows, face powders, transfer tattoos, body hygiene products (i.e., products which do not relate to the care, make-up, or treatment of keratin materials) such as deodorant e.g., in stick form, shampoos, conditioners and make-up products for the eyes such as eyeliners, eye pencils and mascaras, e.g., in cake form, as well as make-up and care products for superficial body growths, for instance keratinous fibers such as the hair, the eyelashes, and the eyebrows or nails.

Another aspect of the invention is a composition comprising at least one liquid fatty phase comprising (i) at least one oil structured with at least one structuring polymer consisting of a polymer (homopolymer or copolymer) with a weight-average molecular mass ranging from 500 to 500,000, containing at least one moiety comprising:

at least one polyorganosiloxane group, consisting of from 1 to 1,000 organosiloxane units in the chain of the moiety or in the form of a graft, and

at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,

the polymer being solid at room temperature and soluble in said oil at a temperature of from 25 to 250° C., and

(ii) at least one film-forming agent,

said oil having an affinity with said structuring polymer and optionally with the film-forming agent, and

the liquid fatty phase, the polymer and the film-forming agent forming a physiologically acceptable medium.

In one embodiment, the composition of the invention comprises at least one aqueous phase. In that case, the composition can be in the form of a dispersion of the aqueous phase in the liquid fatty phase or in the form of a dispersion of the liquid fatty phase in the aqueous phase. In one embodiment, the composition of the invention is in the form of an emulsion. In one embodiment, the aqueous phase can contain a compound soluble in water such as a monoalcohol having 2 to 8 carbon atoms, a polyol or acetone.

In one embodiment, the film-forming agent is a film-forming polymer that can be an organic or inorganic polymer.

In one embodiment, the film-forming agent is a film-forming organic polymer that is solid at room temperature. In one embodiment, the film-forming organic polymer is at least one polymer chosen from the group comprising:

liposoluble film-forming polymers,

lipodispersible film-forming polymers in the form of non-aqueous dispersions of polymer particles, preferably dispersions in silicone or hydrocarbon oils; in one embodiment, the non-aqueous dispersions of polymer comprise polymer particles stabilised on their surface by at least one stabilising agent; these non-aqueous dispersions are often called “NAD”,

aqueous dispersions of polymer particles, often called “latex”; in that case, the composition must comprise an aqueous phase,

water-soluble film-forming polymers; in that case, the composition must comprise an aqueous phase.

As used herein, the expression “at least one” means one or more and thus includes individual components as well as mixtures/combinations.

As used herein, the expression “affinity” means that the polymer and/or the film-forming agent dissolves in said at least one oil, at a temperature higher than the melting temperature or the softening temperature of the structuring polymer and/or at a temperature higher than the melting temperature or the softening temperature of the film-forming agent

The polymer structuring the liquid fatty phase is solid at room temperature (25° C.) and atmospheric pressure (760 mm Hg) and soluble the oil comprised in the liquid fatty phase at a temperature of from 25 to 250° C.

As used herein, the expression “polymer” means a compound having at least two repeating units, preferably at least three repeating unit, more preferably at least ten repeating unit.

In the composition according to the present invention, the structuring polymer represents 0.5 to 80% by weight, preferably 2 to 60% by weight, more preferably 5 to 40% by weight, of the total weight of the composition.

Moreover, the structuring polymer preferably represents 0.1 to 50% by weight of the weight of the film-forming polymer together with the oil included in the liquid fatty phase.

The liquid fatty phase preferably contains at least 30%, and better still at least 40% by weight of silicone oil.

The composition of the invention can be in the form of a paste, a solid or a more or less viscous cream. It can be a single or multiple emulsion, such as an oil-in-water or water-in-oil emulsion or an oil-in-water-in-oil emulsion, or a water-in-oil-in-water emulsion, or a rigid or soft gel containing an oily continuous phase. For example, the liquid fatty phase can be the continuous phase of the composition. In one embodiment, the composition is anhydrous. In one embodiment, the composition is in a form cast as a stick or in a dish, for example solid, and further example, in the form of an oily rigid gel, such as an anhydrous gel, e.g., an anhydrous stick. In a further embodiment, the composition is in the form of an opaque or translucent rigid gel (depending on the presence or absence of pigments), and in a specific example, the liquid fatty phase forms the continuous phase. In one embodiment, the composition is chosen from molded and poured sticks.

The structuring of the liquid fatty phase can be modified depending on the nature of the structuring polymer that is used, and may be such that a rigid structure in the form of a stick is obtained. When these sticks are colored, they make it possible, after application, to obtain a uniformly colored and glossy deposit, which does not migrate, and/or which has good staying power, in particular of the color over time, and/or which does not transfer.

The composition of the invention can be a composition for the lips, such as a lipstick composition in stick form or a composition for the skin, such as a foundation.

Structuring Polymer of the Polyorganosiloxane Type

The polymers used as structuring agents in the composition of the invention are polymers of the polyorganosiloxane type such as those described in documents U.S. Pat. Nos. 5,874,069, 5,919,441, 6,051,216, 5,981,680 and 6,051,216. Nevertheless, these documents specifically deal with deodorant and antiperspirant compositions.

According to the invention, the polymers used as structuring polymer may belong to the following two families:

1) polyorganosiloxanes comprising at least two groups capable of establishing hydrogen interactions, these two groups being located in the polymer chain; and/or

2) polyorganosiloxanes comprising at least two groups capable of establishing hydrogen interactions, these two groups being located on grafts or branches.

The polymers to which the invention applies are solids that may be dissolved beforehand in a solvent with hydrogen interaction capable of breaking the hydrogen interactions of the polymers, for instance C₂ to C₈ lower alcohols and especially ethanol, n-propanol or isopropanol, before being placed in the presence of the hydrophobe oils according to the invention. It is also possible to use these hydrogen interaction “breaking” solvents as co-solvents. These solvents may then be stored in the composition or may be removed by selective evaporation, which is well known to those skilled in the art.

The polymers comprising two groups capable of establishing hydrogen interactions in the polymer chain may be polymers comprising at least one moiety corresponding to the formula:

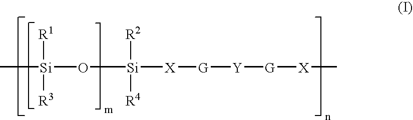

in which:

1) R¹, R², R³ and R⁴, which may be identical or different, represent a group chosen from:

linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms,

C₆ to C₁₀ aryl groups, optionally substituted with one or more C₁ to C₄ alkyl groups,

polyorganosiloxane chains possibly containing one or more oxygen, sulphur and/or nitrogen atoms;

2) the groups X, which may be identical or different, represent a linear or branched C₁ to C₃₀ alkylenediyl group, possibly containing in its chain one or more oxygen and/or nitrogen atoms;

3) Y is a saturated or unsaturated, C₁ to C₅₀ linear or branched divalent alkylene, arylene, cycloalkylene, alkylarylene or arylalkylene group, possibly comprising one or more oxygen, sulphur and/or nitrogen atoms, and/or bearing as substituent one of the following atoms or groups of atoms:

fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or

4) Y represents a group corresponding to the formula:

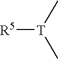

in which

T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and

R⁵ represents a linear or branched C₁ to C₅₀ alkyl group or a polyorganosiloxane chain, possibly comprising one or more ester, amide, urethane, thiocarbamate, urea, thiourea and/or sulphonamide groups, which may be linked to another chain of the polymer;

5) the groups G, which may be identical or different, represent divalent groups chosen from:

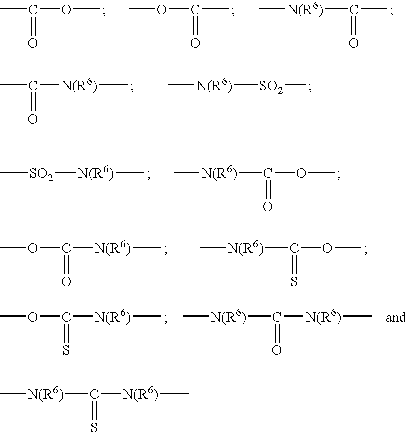

in which R⁶ represents a hydrogen atom or a linear or branched C₁ to C₂₀ alkyl group, on condition that at least 50% of the groups R⁶ of the polymer represents a hydrogen atom and that at least two of the groups G of the polymer are a group other than:

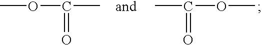

6) n is an integer ranging from 2 to 500 and preferably from 2 to 200, and m is an integer ranging from 1 to 1,000, preferably from 1 to 700 and better still from 6 to 200.

According to the invention, 80% of the groups R¹, R², R³ and R⁴ of the polymer are preferably chosen from methyl, ethyl, phenyl and 3,3,3-trifluoropropyl groups.

According to the invention, Y can represent various divalent groups, furthermore optionally comprising one or two free valencies to establish bonds with other moieties of the polymer or copolymer. Preferably, Y represents a group chosen from:

a) linear C₁ to C₂₀ and preferably C₁ to C₁₀ alkylene groups,

b) C₃₀ to C₅₆ branched alkylene groups possibly comprising rings and unconjugated unsaturations,

c) C₅-C₆ cycloalkylene groups,

d) phenylene groups optionally substituted with one or more C₁ to C₄₀ alkyl groups,

e) C₁ to C₂₀ alkylene groups comprising from 1 to 5 amide groups,

f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,

g) polyorganosiloxane chains of formula:

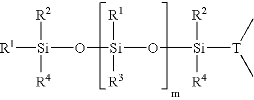

in which R¹, R², R³, R⁴, T and m are as defined above, and

h) polyorganosiloxane chains of formula:

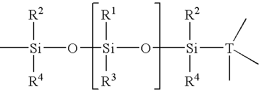

in which R¹, R², R³, R⁴, T and m are as defined above.

The polyorganosiloxanes of the second family may be polymers comprising at least one moiety corresponding to formula (II):

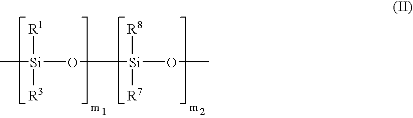

in which

R¹ and R³, which may be identical or different, are as defined above for formula (I),

R⁷ represents a group as defined above for R¹ and R³, or represents a group of formula —X—G—R⁹ in which X and G are as defined above for formula (I) and R⁹ represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,

R⁸ represents a group of formula —X—G—R⁹ in which X, G and R⁹ are as defined above,

m₁ is an integer ranging from 1 to 998, and

m₂ is an integer ranging from 2 to 500.

According to the invention, the polymer used as structuring agent may be a homopolymer, that is to say a polymer comprising several identical moieties, in particular moieties of formula (I) or of formula (II).

According to the invention, it is also possible to use a polymer consisting of a copolymer comprising several different moieties of formula (i), that is to say a polymer in which at least one of the groups R¹, R², R³, R⁴, X, G, Y, m and n is different in one of the moieties. The copolymer may also be formed from several moieties of formula (II), in which at least one of the groups R¹, R³, R⁷, R⁸, m₁ and m₂ is different in at least one of the moieties.

It is also possible to use a copolymer comprising at least one moiety of formula (I) and at least one moiety of formula (II), the moieties of formula (I) and the moieties of formula (II) possibly being identical to or different from each other.

According to one variant of the invention, it is also possible to use a copolymer furthermore comprising at least one hydrocarbon-based moiety comprising two groups capable of establishing hydrogen interactions, chosen from ester, amide, sulphonamide, carbamate, thiocarbamate, urea and thiourea groups, and combinations thereof.

These copolymers may be block copolymers or grafted copolymers.

According to a first embodiment of the invention, the groups capable of establishing hydrogen interactions are amide groups of formulae —C(O)NH— and —HN—C(O)—.

In this case, the structuring polymer may be a polymer comprising at least one moiety of formula (III) or (IV):

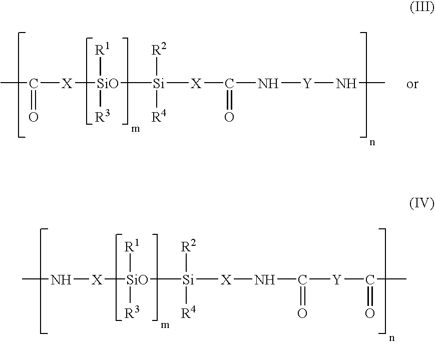

in which R¹, R², R³, R⁴, X, Y, m and n are as defined above.

Such a moiety may be obtained:

either by a condensation reaction between a silicone containing α,ω-carboxylic acid ends and one or more diamines, according to the following reaction scheme:

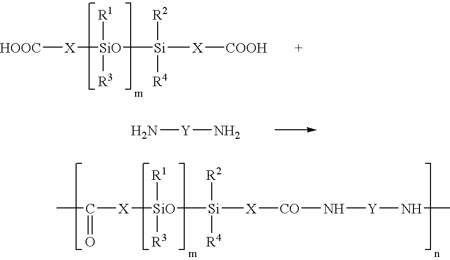

or by reaction of two molecules of α-unsaturated carboxylic acid with a diamine according to the following reaction scheme:

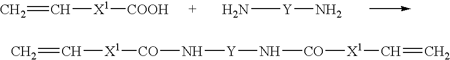

followed by the addition of a siloxane to the ethylenic unsaturations, according to the following scheme:

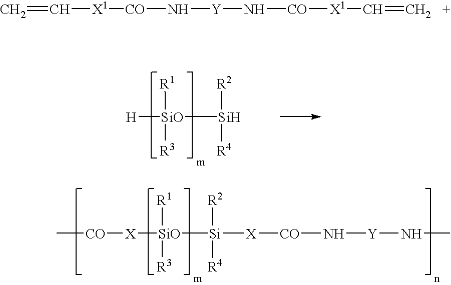

in which X¹—(CH₂)₂— corresponds to X defined above and Y, R¹, R², R³, R⁴ and m are as defined above;

or by reaction of a silicone containing α,ω-NH₂ ends and a diacid of formula HOOC—Y—COOH according to the following reaction scheme:

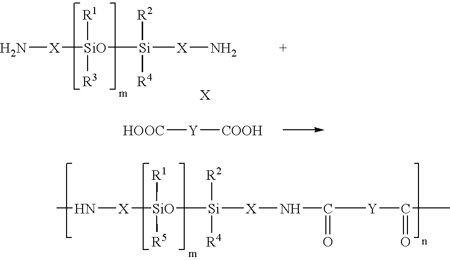

In these polyamides of formula (III) or (IV), m is preferably in the range from 1 to 700, more preferably from 15 to 500 and better still from 15 to 45, and n is in particular in the range from 1 to 500, preferably from 1 to 100 and better still from 4 to 25,

X is preferably a linear or branched alkylene chain containing from 1 to 30 carbon atoms and in particular 3 to 10 carbon atoms, and

Y is preferably an alkylene chain that is linear or branched or that possibly comprises rings and/or unsaturations, containing from 1 to 40 carbon atoms, in particular from 1 to 20 carbon atoms and better still from 2 to 6 carbon atoms, in particular 6 carbon atoms.

In formulae (III) and (IV), the alkylene group representing X or Y can optionally contain in its alkylene portion at least one of the following elements:

1°) 1 to 5 amide, urea or carbamate groups,

2°) a C₅ or C₆ cycloalkyl group, and

3°) a phenylene group optionally substituted with 1 to 3 identical or different C₁ to C₃ alkyl groups.

In formulae (III) and (IV), the alkylene groups may also be substituted with at least one element chosen from the group consisting of:

a hydroxyl group,

a C₃ to C₈ cycloalkyl group,

one to three C₁ to C₄₀ alkyl groups,

a phenyl group optionally substituted with one to three C₁ to C₃ alkyl groups,

a C₁ to C₃ hydroxyalkyl group, and

a C₁ to C₆ aminoalkyl group.

In these formulae (III) and (IV), Y may also represent:

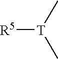

in which R⁵ represents a polyorganosiloxane chain and T represents a group of formula:

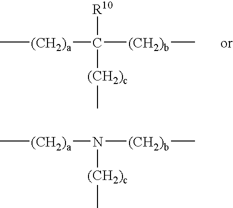

in which a, b and c are, independently, integers ranging from 1 to 10, and R¹⁰ is a hydrogen atom or a group such as those defined for R¹, R², R³ and R⁴.

In formulae (III) and (IV), R¹, R², R³ and R⁴ preferably represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As has been seen previously, the polymer may comprise identical or different moieties of formula (III) or (IV). Thus, the polymer may be a polyamide containing several moieties of formula (III) or (IV) of different lengths, i.e. a polyamide corresponding to the formula:

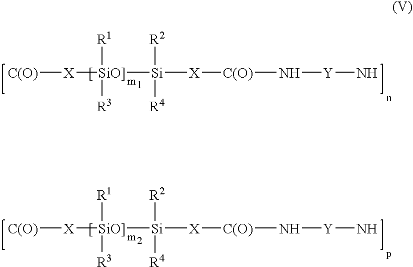

in which X, Y, n and R¹ to R⁴ have the meanings given above, m₁ and m₂, which are different, are chosen in the range from 1 to 1,000, and p is an integer ranging from 2 to 300.

In this formula, the moieties may be structured to form either a block copolymer, or a random copolymer or an alternating copolymer. In this copolymer, the moieties may be not only of different lengths, but also of different chemical structures, for example containing different groups Y. In this case, the copolymer may correspond to the formula:

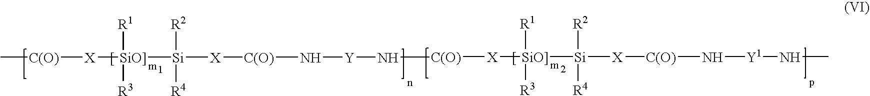

in which R¹ to R⁴, X, Y, m₁, m₂, n and p have the meanings given above and Y¹ is different from Y but chosen from the groups defined for Y. As previously, the various moieties may be structured to form either a block copolymer, or a random copolymer or an alternating copolymer.

In this first embodiment of the invention, the structuring polymer may also consist of a grafted copolymer. Thus, the polyamide containing silicone units may be grafted and optionally crosslinked with silicone chains containing amide groups. Such polymers may be synthesized with trifunctional amines.

In this case, the copolymer may comprise at least one moiety of formula:

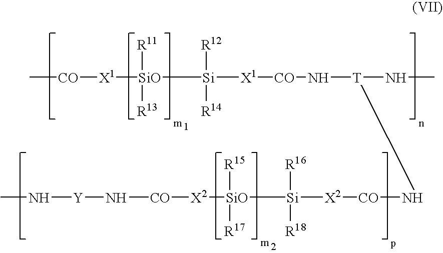

in which X¹ and X², which may be identical or different, have the meaning given for X in formula (I), n is as defined in formula (I), Y and T are as defined in formula (I), R¹¹ to R¹⁸ are groups chosen from the same group as R¹ to R⁴, m₁ and m₂ are numbers in the range from 1 to 1,000, and p is an integer ranging from 2 to 500.

In formula (VII), it is preferred that

p is in the range from 1 to 25 and better still from 1 to 7,

R¹¹ to R¹⁸ are methyl groups,

T corresponds to one of the following formulae:

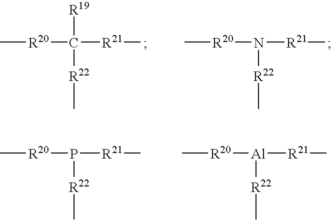

in which R¹⁹ is a hydrogen atom or a group chosen from the groups defined for R¹ to R⁴, and R²⁰, R²¹ and R²² are, independently, linear or branched alkylene groups, and more preferably corresponds to the formula:

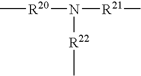

in particular with R²⁰, R²¹ and R²² representing —CH₂—CH₂—,

m₁ and m₂ are in the range from 15 to 500 and better still from 15 to 45,

X¹ and X² represent —(CH₂)₁₀—, and

Y represents —CH₂—.

These polyamides containing a grafted silicone moiety of formula (VII) may be copolymerized with polyamide-silicones of formula (II) to form block copolymers, alternating copolymers or random copolymers. The weight percentage of grafted silicone moieties (VII) in the copolymer may range from 0.5% to 30% by weight.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to the invention, the preferred siloxane-based polyamides are:

polyamides of formula (III) in which m is from 15 to 50;

mixtures of two or more polyamides in which at least one polyamide has a value of m in the range from 15 to 50 and at least one polyamide has a value of m in the range from 30 to 50;

polymers of formula (V) with m₁ chosen in the range from 15 to 50 and m₂ chosen in the range from 30 to 500 with the portion corresponding to m₁ representing 1% to 99% by weight of the total weight of the polyamide and the corresponding portion m₂ representing 1% to 99% by weight of the total weight of the polyamide;

mixtures of polyamide of formula (III) combining

1) 80% to 99% by weight of a polyamide in which n is equal to 2 to 10 and in particular 3 to 6, and

2) 1% to 20% of a polyamide in which n is in the range from 5 to 500 and in particular from 6 to 100;

polyamides corresponding to formula (VI) in which at least one of the groups Y and Y¹ contains at least one hydroxyl substituent;

polyamides of formula (III) synthesized with at least one portion of an activated diacid (diacid chloride, dianhydride or diester) instead of the diacid;

polyamides of formula (III) in which X represents —(CH₂)₃— or —(CH₂)₁₀; and

polyamides of formula (III) in which the polyamides end with a monofunctional chain chosen from the group consisting of monofunctional amines, monofunctional acids, monofunctional alcohols, including fatty acids, fatty alcohols and fatty amines, such as, for example, octylamine, octanol, stearic acid and stearyl alcohol.

According to the invention, the end groups of the polymer chain may end with:

a C₁ to C₅₀ alkyl ester group by introducing a C₁ to C₅₀ monoalcohol during the synthesis,

a C₁ to C₅₀ alkylamide group by taking as stopping group a monoacid if the silicone is α,ω-diaminated, or a monoamine if the silicone is an α,ω-dicarboxylic acid.

According to one embodiment variant of the invention, it is possible to use a copolymer of silicone polyamide and of hydrocarbon-based polyamide, i.e. a copolymer comprising moieties of formula (III) or (IV) and hydrocarbon-based polyamide moieties. In this case, the polyamide-silicone moieties may be arranged at the ends of the hydrocarbon-based polyamide.

Polyamide-based structuring polymers containing silicones may be produced by silylic amidation of polyamides based on fatty acid dimer. This approach involves the reaction of free acid sites existing on a polyamide as end sites, with organosiloxane-monoamines and/or organosiloxane-diamines (amidation reaction), or alternatively with oligosiloxane alcohols or oligosiloxane diols (esterification reaction). The esterification reaction requires the presence of acid catalysts, as is known in the art. It is desirable for the polyamide containing free acid sites, used for the amidation or esterification reaction, to have a relatively high number of acid end groups (for example polyamides with high acid numbers, for example from 15 to 20).

For the amidation of the free acid sites of the hydrocarbon-based polyamides, siloxane diamines with 1 to 300, more particularly 2 to 50 and better still 2, 6, 9.5, 12, 13.5, 23 or 31 siloxane groups, may be used for the reaction with hydrocarbon-based polyamides based on fatty acid dimers. Siloxane diamines containing 13.5 siloxane groups are preferred, and the best results are obtained with the siloxane diamine containing 13.5 siloxane groups and polyamides containing high numbers of carboxylic acid end groups.

The reactions may be carried out in xylene to extract the water produced from the solution by azeotropic distillation, or at higher temperatures (about 180 to 200° C.) without solvent. Typically, the efficacy of the amidation and the reaction rates decrease when the siloxane diamine is longer, that is to say when the number of siloxane groups is higher. Free amine sites may be blocked after the initial amidation reaction of the diaminosiloxanes by reacting them either with a siloxane acid, or with an organic acid such as benzoic acid.

For the esterification of the free acid sites on the polyamides, this may be performed in boiling xylene with about 1% by weight, relative to the total weight of the reagents, of para-toluenesulphonic acid as catalyst.

These reactions carried out on the carboxylic acid end groups of the polyamide lead to the incorporation of silicone moieties only at the ends of the polymer chain.

It is also possible to prepare a copolymer of polyamide-silicone, using a polyamide containing free amine groups, by amidation reaction with a siloxane containing an acid group.

It is also possible to prepare a structuring polymer based on a copolymer between a hydrocarbon-based polyamide and a silicone polyamide, by transamidation of a polyamide having, for example, an ethylene-diamine constituent, with an oligosiloxane-α,ω-diamine, at high temperature (for example 200 to 300° C.), to carry out a transamidation such that the ethylenediamine component of the original polyamide is replaced with the oligosiloxane diamine.

The copolymer of hydrocarbon-based polyamide and of polyamide-silicone may also be a grafted copolymer comprising a hydrocarbon-based polyamide backbone with pendent oligosiloxane groups.

This may be obtained, for example:

by hydrosilylation of unsaturated bonds in polyamides based on fatty acid dimers;

by silylation of the amide groups of a polyamide; or

by silylation of unsaturated polyamides by means of an oxidation, that is to say by oxidizing the unsaturated groups into alcohols or diols, to form hydroxyl groups that are reacted with siloxane carboxylic acids or siloxane alcohols. The olefinic sites of the unsaturated polyamides may also be epoxidized and the epoxy groups may then be reacted with siloxane amines or siloxane alcohols.

According to a second embodiment of the invention, the structuring polymer consists of a homopolymer or a copolymer comprising urethane or urea groups.

As previously, the polymer may comprise polyorganosiloxane moieties containing two or more urethane and/or urea groups, either in the backbone of the polymer or on side chains or as pendent groups.

The polymers comprising at least two urethane and/or urea groups in the backbone may be polymers comprising at least one moiety corresponding to the following formula:

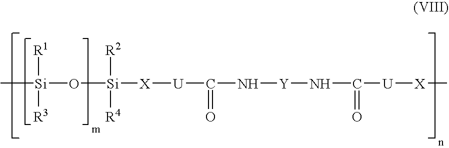

in which R¹, R², R³, R⁴, X, Y, m and n have the meanings given above for formula (I), and U represents —O— or —NH—, such that

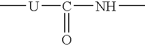

corresponds to a urethane or urea group.

In this formula (VIII), Y may be a linear or branched C₁ to C₄₀ alkylene group, optionally substituted with a C₁ to C₁₅ alkyl group or a C₅ to C₁₀ aryl group. Preferably, a —(CH₂)₆— group is used.

Y may also represent a C₅ to C₁₂ cycloaliphatic or aromatic group that may be substituted with a C₁ to C₁₅ alkyl group or a C₅ to C₁₀ aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane. Generally, it is preferred for Y to represent a linear or branched C₁ to C₄₀ alkylene radical or a C₄ to C₁₂ cycloalkylene radical.

Y may also represent a polyurethane or polyurea block corresponding to the condensation of several diisocyanate molecules with one or more molecules of coupling agents of the diol or diamine type. In this case, Y comprises several urethane or urea groups in the alkylene chain.

It may correspond to the formula:

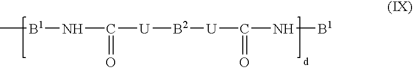

in which B¹ is a group chosen from the groups given above for Y, U is —O— or —NH— and B² is chosen from:

linear or branched C₁ to C₄₀ alkylene groups, which can optionally bear an ionizable group such as a carboxylic acid or sulphonic acid group, or a neutralizable or quaternizable tertiary amine group,

C₅ to C₁₂ cycloalkylene groups, optionally bearing alkyl substituents, for example one to three methyl or ethyl groups, or alkylene, for example the diol radical: cyclohexanedimethanol,

phenylene groups that may optionally bear C₁ to C₃ alkyl substituents, and

groups of formula:

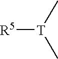

in which T is a hydrocarbon-based trivalent radical possibly containing one or more hetero atoms such as oxygen, sulphur and nitrogen and R⁵ is a polyorganosiloxane chain or a linear or branched C₁ to C₅₀ alkyl chain.

T can represent, for example:

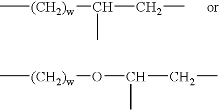

with w being an integer ranging from 1 to 10 and R⁵ being a polyorganosiloxane chain.

When Y is a linear or branched C₁ to C₄₀ alkylene group, the —(CH₂)₂— and —(CH₂)₆— groups are preferred.

In the formula given above for Y, d may be an integer ranging from 0 to 5, preferably from 0 to 3 and more preferably equal to 1 or 2.

Preferably, B² is a linear or branched C₁ to C₄₀ alkylene group, in particular —(CH₂)₂— or —(CH₂)₆— or a group:

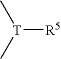

with R⁵ being a polyorganosiloxane chain.

As previously, the polymer constituting the structuring polymer may be formed from silicone urethane and/or silicone urea moieties of different length and/or constitution, and may be in the form of block or random copolymers.

According to the invention, the silicone may also comprise urethane and/or urea groups no longer in the backbone but as side branches.

In this case, the polymer may comprise at least one moiety of formula:

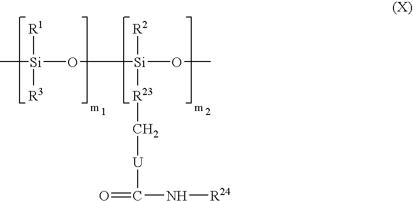

in which R¹, R², R³, m₁ and m₂ have the meanings given above for formula (I),

U represents O or NH,

R²³ represents a C₁ to C₄₀ alkylene group, optionally comprising one or more hetero atoms chosen from O and N, or a phenylene group, and

R²⁴ is chosen from linear, branched or cyclic, saturated or unsaturated C₁ to C₅₀ alkyl groups, and phenyl groups optionally substituted with one to three C₁ to C₃ alkyl groups.

The polymers comprising at least one moiety of formula (X) contain siloxane units and urea or urethane groups, and they may be used as structuring polymer in the compositions of the invention.

The siloxane polymers may have a single urea or urethane group by branching or may have branches containing two urea or urethane groups, or alternatively they may contain a mixture of branches containing one urea or urethane group and branches containing two urea or urethane groups.

They may be obtained from branched polysiloxanes, comprising one or two amino groups by branching, by reacting these polysiloxanes with monoisocyanates.

As examples of starting polymers of this type containing amino and diamino branches, mention may be made of the polymers corresponding to the following formulae:

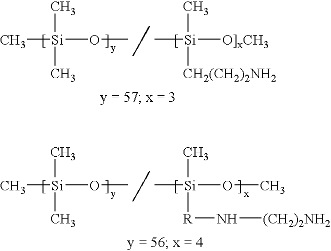

In these formulae, the symbol “/” indicates that the segments may be of different lengths and in a random order, and R represents a linear aliphatic group preferably containing 1 to 6 carbon atoms and better still 1 to 3 carbon atoms.

Such polymers containing branching may be formed by reacting a siloxane polymer, containing at least three amino groups per polymer molecule, with a compound containing only one monofunctional group (for example an acid, an isocyanate or an isothiocyanate) to react this monofunctional group with one of the amino groups and to form groups capable of establishing hydrogen interactions. The amino groups may be on side chains extending from the main chain of the siloxane polymer, such that the groups capable of establishing hydrogen interactions are formed on these side chains, or alternatively the amino groups may be at the ends of the main chain, such that the groups capable of hydrogen interaction will be end groups of the polymer.

As a procedure for forming a polymer containing siloxane units and groups capable of establishing hydrogen interactions, mention may be made of the reaction of a siloxane diamine and of a diisocyanate in a silicone solvent so as to provide a gel directly. The reaction may be performed in a silicone fluid, the resulting product being dissolved in the silicone fluid, at high temperature, the temperature of the system then being reduced to form the gel.

The polymers that are preferred for incorporation into the compositions according to the present invention are siloxane-urea copolymers that are linear and that contain urea groups as groups capable of establishing hydrogen interactions in the backbone of the polymer.

As an illustration of a polysiloxane ending with four urea groups, mention may be made of the polymer of formula:

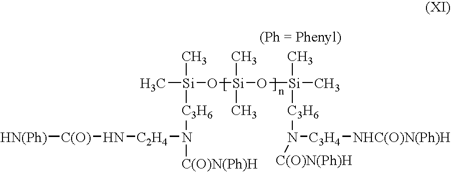

in which Ph is a phenyl group and n is a number from 0 to 300, in particular from 0 to 100, for example 50.

This polymer is obtained by reacting the following polysiloxane containing amino groups:

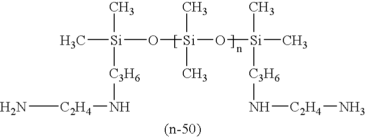

with phenyl isocyanate.

The polymers of formula (VIII) comprising urea or urethane groups in the chain of the silicone polymer may be obtained by reaction between a silicone containing α,ω—NH₂ or —OH end groups, of formula:

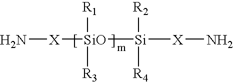

in which m, R¹, R², R³, R⁴ and X are as defined for formula (I) and a diisocyanate OCN—Y—NCO in which Y has the meaning given in formula (I); and optionally a diol or diamine coupling agent of formula H₂N—B²—NH₂ or HO—B²—OH, in which B² is as defined in formula (IX).

According to the stoichiometric proportions between the two reagents, diisocyanate and coupling agent, Y may have the formula (IX) with d equal to 0 or d equal to 1 to 5.

As in the case of the polyamide silicones of formula (II) or (III), it is possible to use in the invention polyurethane or polyurea silicones containing moieties of different length and structure, in particular moieties whose lengths differ by the number of silicone units. In this case, the copolymer may correspond, for example, to the formula:

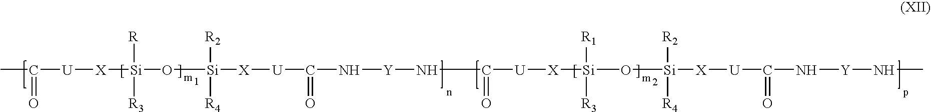

in which R¹, R², R³, R⁴, X, Y and U are as defined for formula (VIII) and m₁, m₂, n and p are as defined for formula (V).

Branched polyurethane or polyurea silicones may also be obtained using, instead of the diisocyanate OCN—Y—NCO, a triisocyanate of formula:

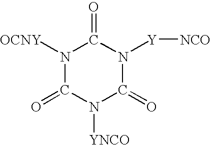

A polyurethane or polyurea silicone containing branches comprising an organosiloxane chain with groups capable of establishing hydrogen interactions is thus obtained. Such a polymer comprises, for example, a moiety corresponding to the formula:

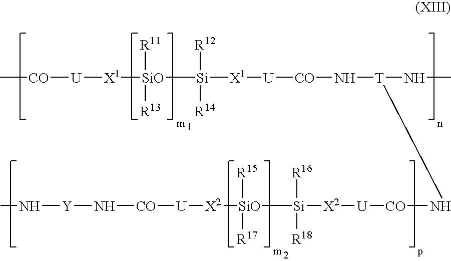

in which X¹ and X², which are identical or different, have the meaning given for X in formula (I), n is as defined in formula (I), Y and T are as defined in formula (I), R¹¹ to R¹⁸ are groups chosen from the same group as R¹ to R⁴, m₁ and m₂ are numbers in the range from 1 to 1,000, and p is an integer ranging from 2 to 500.

As in the case of the polyamides, this copolymer can also comprise polyurethane silicone moieties without branching.

In this second embodiment of the invention, the siloxane-based polyureas and polyurethanes that are preferred are:

polymers of formula (VIII) in which m is from 15 to 50;

mixtures of two or more polymers in which at least one polymer has a value of m in the range from 15 to 50 and at least one polymer has a value of m in the range from 30 to 50;

polymers of formula (XII) with m₁ chosen in the range from 15 to 50 and m₂ chosen in the range from 30 to 500 with the portion corresponding to m₁ representing 1% to 99% by weight of the total weight of the polymer and the portion corresponding to m₂ representing 1% to 99% by weight of the total weight of the polymer;

mixtures of polymer of formula (VIII) combining

1) 80% to 99% by weight of a polymer in which n is equal to 2 to 10 and in particular 3 to 6, and

2) 1% to 20% of a polymer in which n is in the range from 5 to 500 and in particular from 6 to 100,

copolymers comprising two moieties of formula (VIII) in which at least one of the groups Y contains at least one hydroxyl substituent;

polymers of formula (VIII) synthesized with at least one portion of an activated diacid (diacid chloride, dianhydride or diester) instead of the diacid;

polymers of formula (VIII) in which X represents —(CH₂)₃— or —(CH₂)₁₀—; and

polymers of formula (VIII) in which the polymers end with a multifunctional chain chosen from the group consisting of monofunctional amines, monofunctional acids, monofunctional alcohols, including fatty acids, fatty alcohols and fatty amines, such as, for example, octylamine, octanol, stearic acid and stearyl alcohol.

As in the case of the polyamides, copolymers of polyurethane or polyurea silicone and of hydrocarbon-based polyurethane or polyurea may be used in the invention by performing the reaction for synthesizing the polymer in the presence of an α,ω-difunctional block of non-silicone nature, for example a polyester, a polyether or a polyolefin.

As has been seen previously, the structuring polymer consisting of homopolymers or copolymers of the invention may contain siloxane moieties in the main chain of the polymer and groups capable of establishing hydrogen interactions, either in the main chain of the polymer or at the ends thereof, or on side chains or branches of the main chain. This may correspond to the following five arrangements:

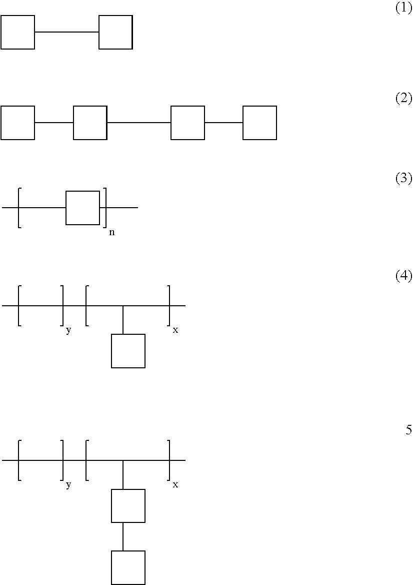

in which the continuous line is the main chain of the siloxane polymer and the squares represent the groups capable of establishing hydrogen interactions.

In case (1), the groups capable of establishing hydrogen interactions are arranged at the ends of the main chain.

In case (2), two groups capable of establishing hydrogen interactions are arranged at each of the ends of the main chain.

In case (3), the groups capable of establishing hydrogen interactions are arranged within the main chain in repeating moieties.

In cases (4) and (5), these are copolymers in which the groups capable of establishing hydrogen interactions are arranged on branches of the main chain of a first series of moieties that are copolymerized with moieties not comprising groups capable of establishing hydrogen interactions. The values n, x and y are such that the polymer has the desired properties in terms of a structuring polymer.

According to the invention, the structuring of the liquid fatty phase containing at least one silicone oil is obtained with the aid of one or more of the polymers mentioned above, in combination with solid particles with a hydrophobic surface.

As examples of polymers that may be used, mention may be made of the silicone polyamides obtained in accordance with Examples 1 and 2 of document U.S. Pat. No. 5,981,680.

The at least one structuring polymer in the compositions of the invention may have a softening point greater than 50° C., such as from 65° C. to 190° C. and for example less than 150° C. and further such as from 70° C. to 130° C. and even further such as from 80° C. to 105° C. This softening point may be lower than that of structuring polymers used in the art which may facilitate the use of the at least one structuring polymer of the present invention and may limit the degradation of the liquid fatty phase. These polymers may be non-waxy polymers.

The softening point can be measured by a well-known method as “Differential Scanning Calorimetry” (i.e. DSC method) with a temperature rise of 5 to 10° C./min. They have good solubility in the silicone oils and produce macroscopically homogeneous compositions. Preferably, they have an average molecular mass from 500 to 200,000, for example from 1,000 to 100,000 and preferably from 2,000 to 30,000.

In one embodiment, the structuring polymer is a silicone polyamide, also called a polyamide-modified silicone. In another embodiment, the structuring polymer is non soluble in an aqueous phase.

Further, an embodiment of the invention relates to a skin, lips, or keratinous fibers care, treatment, or make-up composition comprising a structured composition containing at least one liquid fatty phase comprising (i) at least one oil structured with at least one above mentioned structuring polymer and (ii) at least one film-forming agent,

said oil having an affinity with said structuring polymer and optionally with the film-forming agent and

the liquid fatty phase, the polymer and the film-forming agent forming a physiologically acceptable medium.

Additionally, an embodiment of the invention relates to a skin, lips, or keratinous fibers care or make-up composition comprising structured composition containing at least one liquid fatty phase comprising (i) at least one oil structured with at least one above mentioned structuring polymer (ii) at least one film-forming agent, and at least one coloring agent,

said oil having an affinity with said structuring polymer and optionally with the film-forming agent, and

the liquid fatty phase, the polymer and the film-forming agent forming a physiologically acceptable medium.

Another embodiment of the invention relates to a mascara, an eyeliner, a foundation, a lipstick, a blusher, a make-up-removing product, a make-up product for the body, an eyeshadow, a face powder, a concealer product, a shampoo, a conditioner, an antisun product or a care product for the lips, skin, or hair comprising a composition comprising at least one liquid fatty phase in the mascara, eyeliner, foundation, lipstick, blusher, make-up-removing product, make-up product for the body, eyeshadow, face powder, concealer product, shampoo, conditioner, antisun product or care product for the skin, lips, or hair which comprises at least one liquid fatty phase comprising (i) at least one oil structured with at least one above mentioned structuring polymer and (ii) at least one film-forming agent,

said oil having an affinity with said structuring polymer and optionally with the film-forming agent and

the liquid fatty phase, the polymer and the film-forming agent forming a physiologically acceptable medium.

Another embodiment of the invention relates to a deodorant product or a care product for the skin or body comprising a composition containing at least one liquid fatty phase comprising (i) at least one oil structured with at least one above mentioned structuring polymer and (ii) at least one polymeric film-forming agent,

said oil having an affinity with said structuring polymer and optionally with the film-forming agent and

the liquid fatty phase, the polymer and the film-forming agent forming a physiologically acceptable medium.

In one embodiment, this composition is anhydrous and the film-forming polymer is a film-forming liposoluble or lipodispersible polymer or a mixture thereof.

Another embodiment of the invention relates to a lipstick composition comprising an anhydrous composition containing at least one liquid fatty phase comprising (i) at least one oil structured with at least one above mentioned structuring polymer and (ii) at least one film-forming agent,

said oil having an affinity with said structuring polymer and optionally with the film-forming agent, and

the liquid fatty phase, the polymer and the film-forming agent forming a physiologically acceptable medium.

In one embodiment, this lipstick is anhydrous and contains at least one film-forming liposoluble or lipodispersible polymer.

Another embodiment of the invention relates to a method for care, make-up or treatment of keratin materials comprising applying to the keratin materials a composition containing at least one liquid fatty phase comprising (i) at least one oil structured with at least one above mentioned structuring polymer and (ii) at least one film-forming agent,

said oil having an affinity with said structuring polymer and optionally with the film-forming agent and

the liquid fatty phase, the polymer and the film-forming agent forming a physiologically acceptable medium.

In one embodiment, this composition is anhydrous and contains at least one film-forming liposoluble or lipodispersible polymer.

Another embodiment of the invention relates to a method for care, make-up or treatment of keratinous fibers, lips, or skin comprising applying to the keratinous fibers, lips, or skin a composition comprising at least one liquid fatty phase comprising (i) at least one oil structured with at least one above mentioned structuring polymer and (ii) at least one film-forming agent,

said oil having an affinity with said structuring polymer and optionally with the film-forming agent and

the liquid fatty phase, the polymer and the film-forming agent forming a physiologically acceptable medium.

Another embodiment of the invention relates to a method for providing an anhydrous composition having at least one property chosen from non-exudation, gloss, and comfortable deposit on keratin materials chosen from lips, skin, and keratinous fibers, comprising including in the composition at least one liquid fatty phase comprising

(i) at least one oil structured with at least one above mentioned structuring polymer and

(ii) at least one film-forming agent,

said oil having an affinity with said structuring polymer and optionally with the film-forming agent and

the liquid fatty phase, the polymer and the film-forming agent forming a physiologically acceptable medium.

Another embodiment of the invention relates to a method of making up or caring for skin, lips or keratinous fibers comprising applying to the skin, lips, or keratinous fibers a structured composition containing at least one liquid fatty phase comprising (i) at least one oil structured with at least one above mentioned structuring polymer and (ii) at least one film-forming agent,

said oil having an affinity with said structuring polymer and optionally with the film-forming agent, and the liquid fatty phase, the polymer and the film-forming agent forming a physiologically acceptable medium.

Depending on the intended application, such as a stick, hardness of the composition may also be considered. The hardness of a composition may, for example, be expressed in gramforce (gf). The composition of the present invention may, for example, have a hardness ranging from 20 gf to 2000 gf, such as from 20 gf to 900 gf, and further such as from 20 gf to 600 gf.

This hardness is measured in one of two ways. A first test for hardness is according to a method of penetrating a probe into the composition and in particular using a texture analyzer (for example TA-XT2i from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf, such as from 30 gf to 250 gf, for a sample of 8.1 mm in diameter stick, and further such as from 30 gf to 200 gf, and also further such as from 30 gf to 120 gf for a sample of 12.7 mm in diameter stick.

The hardness of the composition of the present invention may be such that the compositions are self-supporting and can easily disintegrate to form a satisfactory deposit on a keratinous material. In addition, this hardness may impart good impact strength to the inventive compositions, which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention.

As is evident, the hardness of the composition according to the invention may, for example, be such that the composition is advantageously self-supporting and can disintegrate easily to form a satisfactory deposit on the skin and/or the lips and/or superficial body growths, such as keratinous fibers. In addition, with this hardness, the composition of the invention may have good impact strength.

According to the invention, the composition in stick form may have the behavior of a deformable, flexible elastic solid, giving noteworthy elastic softness on application. The compositions in stick form of the prior art do not have these properties of elasticity and flexibility.

Another embodiment of this invention relates to a skin composition as for example a sun-care product or a foundation for the human face or body, comprising a composition containing at least one liquid fatty phase comprising (i) at least one oil structured with at least one above mentioned structuring polymer and (ii) at least one film-forming agent,

said oil having an affinity with said structuring polymer and optionally with the film-forming agent, and

the liquid fatty phase, the polymer and the film-forming agent forming a physiologically acceptable medium.

In one embodiment, the skin composition comprises an aqueous phase.

Another embodiment of the invention relates to a mascara or hair composition comprising a composition containing at least one liquid fatty phase comprising (i) at least one oil structured with at least one above mentioned structuring polymer and (ii) at least one film-forming agent,

said oil having an affinity with said structuring polymer and optionally with the film-forming agent, and

the liquid fatty phase, the polymer and the film-forming agent forming a physiologically acceptable medium.

Liquid Fatty Phase

For the purposes of the invention, the expression “liquid fatty phase” means a fatty phase which is liquid at room temperature (25° C.) and atmospheric pressure (760 mmHg, i.e. 101 KPa), composed of one or more fatty substances that are liquid at room temperature, also referred to as oils, that are generally mutually compatible, i.e. forming a homogeneous phase macroscopically. The expression “liquid fatty substance” means a non-aqueous liquid medium which is immiscible in all proportions with water, for example, a hydrocarbon-based compound comprising one or more carbon chains each containing at least 5 carbon atoms and possibly comprising at least one polar group chosen from carboxylic acid, hydroxyl, polyol, amine, amide, phosphoric acid, phosphate, ester, ether, urea, carbamate, thiol, thioether and thioester, a silicone compound optionally comprising carbon chains at the end or pendant, these chains optionally being substituted with a group chosen from fluoro, perfluoro, (poly)amino acid, ether, hydroxyl, amine, acid and ester groups; or a fluoro or perfluoro compound such as fluorohydrocarbons or perfluorohydrocarbons containing at least 5 carbon atoms, possibly comprising a hetero atom chosen from N, O, S and P and optionally at least one function chosen from ether, ester, amine, acid, carbamate, urea, thiol and hydroxyl groups.

The at least one liquid fatty phase, in one embodiment, may comprise at least one oil having an affinity with the structuring polymer and optionally with the film-forming polymer. The at least one oil, for example, may be chosen from polar oils and apolar oils including hydrocarbon-based liquid oils and oily liquids at room temperature. In one embodiment, the composition of the invention comprises at least one structuring polymer, at least one film-forming agent and at least one apolar oil. The polar oils of the invention, for example, may be added to an apolar oil, the apolar oils acting in particular as co-solvent for the polar oils.

The liquid fatty phase of the composition may contain more than 30%, for example, more than 40%, of liquid oil(s) containing a group similar to that of the units of the structuring polymer, and for example from 50% to 100%. In one embodiment, the liquid fatty phase structured with a silicone-polyamide-type skeleton contains a high quantity, ie., greater than 30%, for example greater than 40% relative to the total weight of the liquid fatty phase, or from 50% to 100%, of at least one apolar, such as hydrocarbon-based oil, silicone oils or mixtures thereof. For the purposes of the invention, the expression “hydrocarbon-based oil” means an oil essentially comprising carbon and hydrogen atoms, optionally with at least one group chosen from hydroxyl, ester, carboxyl and ether groups. With such a fatty phase, the at least one film-forming agent may, for example, contain an amine, amide, urethane or silicone group.

For a liquid fatty phase structured with a polymer containing a partially silicone-based skeleton, this fatty phase may contain more than 30%, for example, more than 40%, relative to the total weight of the liquid fatty phase and, for example, from 50% to 100%, of at least one silicone-based liquid oil, relative to the total weight of the liquid fatty phase. In this embodiment, the at least one film-forming agent may comprise a silicone group.

For example, the at least one polar oil useful in the invention may be chosen from:

hydrocarbon-based plant oils with a high content of triglycerides comprising fatty acid esters of glycerol in which the fatty acids may have varied chain lengths from C₄ to C₂₄, these chains possibly being chosen from linear and branched, and saturated and unsaturated chains; these oils can be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;

synthetic oils or esters of formula R₅COOR₆ in which R₅ is chosen from linear and branched fatty acid residues containing from 1 to 40 carbon atoms and R₆ is chosen from, for example, a hydrocarbon-based chain containing from 1 to 40 carbon atoms, on condition that R₅+R₆≧10, such as, for example, purcellin oil (cetostearyl octanoate), isononyl isononanoate, C₁₂-C₁₅ alkyl benzoates, isopropyl myristate, 2-ethylhexyl palmitate, isostearyl isostearate and alkyl or polyalkyl octanoates, decanoates or ricinoleates; hydroxylated esters such as isostearyl lactate and diisostearyl malate; and pentaerythritol esters;

synthetic ethers containing from 10 to 40 carbon atoms;

C₈ to C₂₆ fatty alcohols such as oleyl alcohol; and

C₈ to C₂₆ fatty acids such as oleic acid, linolenic acid or linoleic acid.

The at least one apolar oil according to the invention is chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof, liquid petrolatum, liquid lanolin, polydecenes, hydrogenated polyisobutene such as Parleam®, and squalane; and mixtures thereof. The structured oils, for example those structured with silicone polyamides may be, in one embodiment, apolar oils, such as silicone oils.

In one embodiment, the liquid fatty phase comprises one or more silicone oils, in particular at least one non-volatile oil chosen from phenylsilicones such as phenyl trimethicones.

In another embodiment, the viscosity of the oil according to the invention, in particular silicone oil, is less than 1000 cSt, and for example less than 100 cSt.

In another embodiment, the liquid fatty phase comprises one or more volatile oils chosen from silicone oils. In one embodiment, the volatile silicone oil is chosen from polydimethylsiloxanes, linear or cyclic, having 2 to 7 silicium atoms and optionally having an alkyl group or an alkoxy group having 2 to 10 carbon atoms.

For the purposes of the invention, the expression “volatile solvent or oil” means any non-aqueous medium capable of evaporating on contact with the skin or the lips in less than one hour at room temperature and atmospheric pressure. The volatile solvent(s) of the invention is(are) organic solvents, such as volatile cosmetic oils that are liquid at room temperature, having a non-zero vapor pressure, at room temperature and atmospheric pressure, ranging in particular from 10⁻² to 300 mmHg (1.33 to 40,000 Pa) and, for example, greater than 0.03 mmHg (4 Pa) and further example greater than 0.3 mmHg (40 Pa). The expression “non-volatile oil” means an oil which remains on the skin or the lips at room temperature and atmospheric pressure for at least several hours, such as those having a vapor pressure of less than 10⁻² mmHg (1.33 Pa).

According to the invention, these volatile solvents may facilitate the staying power or long wearing properties of the composition on the skin, the lips or superficial body growths such as nails and keratinous fibers. The solvents can be chosen from hydrocarbon-based solvents, silicone solvents optionally comprising alkyl or alkoxy groups that are pendant or at the end of a silicone chain, and a mixture of these solvents.

The volatile oil(s), in one embodiment, can be present in an amount ranging from 0% to 95.5% relative to the total weight of the composition, such as from 2% to 75% or, for example, from 10% to 45%. This amount will be adapted by a person skilled in the art according to the desired staying power or long wearing properties.

In practice, the total liquid fatty phase can be, for example, present in an amount ranging from 1% to 99% by weight relative to the total weight of the composition, for example from 5% to 99%, 5% to 95.5%, from 10% to 80% or from 20% to 75%.

The at least one liquid fatty phase of the composition of the invention may further comprises a dispersion of lipid vesicles. The composition of the invention may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally containing dispersed lipid vesicles, or a fatty phase optionally containing dispersed lipid vesicles. In one embodiment, the composition has a continuous oily phase or fatty phase and is more specifically an anhydrous composition in, for example, a stick or dish form. An anhydrous composition is one that has less than 10% water by weight, such as, for example, less than 5% by weight.

Film-Forming Agent

The composition of the invention also contains at least one film-forming agent, which can be an organic or inorganic polymer. In one embodiment, the film-forming organic polymer is at least one polymer chosen from the group comprising:

liposoluble film-forming polymers,

lipodispersible film-forming polymers in the form of non-aqueous dispersions of polymer particles, preferably dispersions in silicone or hydrocarbon oils; in one embodiment, the non-aqueous dispersions of polymer comprise polymer particles stabilised on their surface by at least one stabilising agent; these non-aqueous dispersions are often called “NAD”,

aqueous dispersions of polymer particles, often called “latex”; in that case, the composition must comprise an aqueous phase,

water-soluble film-forming polymers; in that case, the composition must comprise an aqueous phase.

In one embodiment, the film-forming agent is a film-forming liposoluble or lipodispersible organic polymer.

I/Liposoluble Polymers

They have no, or very little, gelling nature for the oil medium chosen, in particular when they are used at a concentration less than or equal to 50% by weight. The liposoluble polymers may be of any chemical type and include especially:

a) the liposoluble, amorphous homopolymers and copolymers of olefins, cycloolefins, butadiene, isoprene, styrene, ethers, vinyl esters or amides, esters or amides of (meth)acrylic acid containing a linear, branched or cyclic C₄₋₅₀ alkyl group, and preferably amorphous. The preferred liposoluble homopolymers and copolymers are obtained from monomers chosen from within the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tertio-butyl (meth)acrylate, tridecyl (meth)acrylate, stearyl (meth)acrylate, or mixtures thereof. There will be cited, for example, the alkyl acrylate/cycloalkyl acrylate copolymer sold by PHOENIX CHEM. under the name GIOVAREZ AC-5099 ML, and the vinyl pyrrolidone copolymers, such as copolymers of a C₂ to C₃₀, such as C₃ to C₂₂ alkene, and combinations thereof, can be used. As examples of VP copolymers which can be used in the invention, mention may be made of VP/vinyllaurate, VP/vinylstearate butylated polyvinylpyrrolidone (PVP), VP/hexadecene, VP/triacontene or VP/acrylic acid/lauryl methacrylate copolymer.

As special liposoluble copolymers, there may be cited:

i) the grafted acrylic-silicone polymers with a silicone backbone, acrylic grafts or with an acrylic backbone, silicone grafts such as the product sold under the name SA 70.5 by 3M and described in the patents U.S. Pat. Nos. 5,725,882, 5,209,924, 4,972,037, 4,981,903, 4,981,902, 5,468,477 and in the patents U.S. Pat. No. 5,219,560, EP 0 388 582.

ii) the liposoluble polymers bearing fluorous groups belonging to one of the classes described hereinabove, in particular those described in the patent U.S. Pat. No. 5,948,393, the alkyl (meth)acrylate/perfluoroalkyl (meth)acrylate copolymers described in the patents EP 0 815 836 and U.S. Pat. No. 5,849,318.

iii) polymers or copolymers resulting from the polymerization or copolymerization of an ethylenic monomer, comprising one or more ethylenic, preferably conjugated bonds (or dienes). As polymers or copolymers resulting from the polymerization or copolymerization of an ethylenic monomer, use may be made of vinyl, acrylic or methacrylic copolymers which may be block copolymers, such as diblock or triblock copolymers, or even multiblock or starburst or radial copolymers. The at least one ethylenic film-forming agent may comprise, for example, a styrene block (S), an alkylstyrene block (AS), an ethylene/butylene block (EB), an ethylene/propylene block (EP), a butadiene block (B), an isoprene block (I), an acrylate block (A), a methacrylate block (MA) or a combination of these blocks.

In one embodiment, a copolymer comprising at least one styrene block is used as film-forming agent. A triblock copolymer and in particular those of the polystyrene/polyisoprene or polystyrene/ polybutadiene type, such as those sold or made under the name “Luvitol HSB” by BASF and those of the polystyrene/copoly(ethylene-propylene) type or alternatively of the polystyrene/copoly(ethylene/butylene) type, such as those sold or made under the brand name “Kraton” by Shell Chemical Co. or Gelled Permethyl 99A by Penreco, may be used. Styrene-methacrylate copolymers can also be used.

As ethylenical film-forming agent which can be used in the composition of the invention, mention may be made, for example, of Kraton (G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton G1750X (EP) multiarm, Kraton G1765X (EP) multiarm, Kraton D-1101 (SBS), Kraton D-1102 (SBS), Kraton D-1107 (SIS), Gelled Permethyl 99A -750, Gelled Permethyl 99A-753-58 (mixture of starburst block polymer and triblock polymer), Gelled Permethyl 99A-753-59 (mixture of starburst block polymer and triblock polymer), Versagel 5970 and Versagel 5960 from Penreco (mixture of starburst polymer and triblock polymer in isododecane), and OS 129880, OS 129881 and OS 84383 from Lubrizol (styrene-methacrylate copolymer). As examples of liposoluble polymers which can be used in the invention, mention may be made of polyalkylenes, in particular polybutene.

b) the liposoluble, amorphous polycondensates, preferably not comprising hydrogen-interaction donor groups, in particular polyesters with C₄₋₅₀ alkyl side chains or else polyesters resulting from the condensation of fatty acid dimers, or even polyesters comprising a silicone segment in the form of a sequence, graft or end group, solid at room temperature ° C., such as defined in the patent application FR 0113920, as yet unpublished.

c) the liposoluble, amorphous polysaccharides comprising alkyl (ether or ester) side chains, in particular alkylcelluloses with a linear or branched, saturated or unsaturated C₁ to C₈ alkyl radical, such as ethylcellulose and propylcellulose

As a general rule, the film-forming liposoluble polymers of the invention have a molecular weight ranging between 1,000 and 500,000, preferably between 2,000 and 250,000, and a glass transition temperature ranging between −100° C. and +300° C., preferably between −50° C. and +100° C., preferably still between −10° C. and +90° C.

II/Lipodispersible Polymers: Non-Aqueous Dispersions of Polymer Particles

According to another embodiment, the at least one film-forming polymer can be chosen from stable non-aqueous dispersions of polymer particles, that are generally spherical, of one or more polymers, in a physiologically acceptable liquid fatty phase, such as hydrocarbone-base oils or silicone oils. These dispersions are generally known as NADs (non-aqueous dispersions) of polymer, as opposed to latices, which are aqueous dispersions of polymer. These dispersions may especially be in the form of nanoparticles of polymers in stable dispersion in the said fatty phase. In one embodiment, the nanopartides are between 5 nm and 600 nm in size. However, it is possible to obtain polymer particles ranging up to 1 μm in size.

One advantage of the polymer dispersion of the composition of the invention is the possibility of varying the glass transition temperature (Tg) of the polymer or the polymer system (polymer plus additive of the plasticizer type), and of thus going from a hard polymer to a more or less soft polymer, making it possible to adjust the mechanical properties of the composition depending on the intended application and in particular on the film deposited.

The polymers in dispersion which may be used in the composition of the invention preferably have a molecular weight of about from 2,000 to 10,000,000 and a Tg of from −100° C. to 300° C. and better still from −50° C. to 50° C. and preferably from −10° C. to 100° C.

It is possible to use film-forming polymers, that preferably have a low Tg, of less than or equal to the temperature of the skin and especially less than or equal to 40° C. A dispersion is thus obtained which can form a film when it is applied to a support

Among the film-forming polymers which may be mentioned are free-radical, acrylic or vinyl homopolymers or copolymers, preferably having a Tg of less than or equal to 40° C. and especially ranging from −10° C. to 30° C. used alone or as a mixture.

The expression <<free-radical polymer>>means a polymer obtained by polymerization of monomers containing unsaturation, especially ethylenic unsaturation, each monomer being capable of homopolymerizing (unlike polycondensates). The free-radical polymers may especially be vinyl polymers or copolymers, especially acrylic polymers.

The vinyl polymers may result from the polymerization of ethylenically unsaturated monomers containing at least one acid group and/or esters of these acidic monomers and/or amides of these acids.

As monomers bearing an acidic group, it is possible to use α,β-ethylenic unsaturated carboxylic acids such as acrylic acid, methacrylic acid, crotonic acid, maleic acid or itaconic acid. (Meth)acrylic acid and crotonic acid are preferably used, and more preferentially (meth)acrylic acid.

The esters of acidic monomers are advantageously chosen from the esters of (meth)acrylic acid (also known as (meth)acrylates), for instance alkyl (meth)acrylates, in particular of a C₁-C₂₀ and preferably a C₁-C₈ alkyl, aryl (meth)acrylates, in particular of a C₆-C₁₀ aryl, and hydroxyalkyl (meth)acrylates, in particular of a C₂-C₆ hydroxyalkyl. Alkyl (meth)acrylates which may be mentioned include methyl, ethyl, butyl, isobutyl, 2-ethylhexyl and lauryl (meth)acrylate. Hydroxyalkyl (meth)acrylates which may be mentioned include hydroxyethyl (meth)acrylate and 2-hydroxypropyl (meth)acrylate. Aryl (meth)acrylates which may be mentioned include benzyl or phenyl acrylate.

The (meth)acrylic acid esters that are particularly preferred are the alkyl (meth)acrylates.

Free-radical polymers that are preferably used are copolymers of (meth)acrylic acid and of an alkyl (meth)acrylate, especially of a C₁-C₄ alkyl. More preferentially, methyl acrylates may be used, optionally copolymerized with acrylic acid.

Amides of the acidic monomers which may be mentioned include (meth)acrylamides, and especially N-alkyl(meth)acrylamides, in particular of a C₂-C₁₂ alkyl, such as N-ethylacrylamide, N-t-butylacrylamide and N-octylacrylamide; N-di(C₁-C₄)alkyl(meth)acryl-amides.

The vinyl polymers may also result from the polymerization of ethylenically unsaturated monomers containing at least one amine group, in free form or partially or totally neutralized, or alternatively partially or totally quaternized. Such monomers may be, for example, dimethylaminoethyl (meth)acrylate, dimethylaminoethylmethacrylamide, vinylamine, vinylpyridine and diallyldimethylammonium chloride.

The vinyl polymers may also result from the homopolymerization or copolymerization of at least one monomer chosen from vinyl esters and styrene monomers. In particular, these monomers may be polymerized with acidic monomers and/or esters thereof and/or amides thereof, such as those mentioned above. Examples of vinyl esters which may be mentioned include vinyl acetate, vinyl propionate, vinyl neodecanoate, vinyl pivalate, vinyl benzoate and vinyl t-butylbenzoate. Styrene monomers, which may be mentioned, include styrene and α-methylstyrene.

The list of monomers given is not limiting, and it is possible to use any monomer known to those skilled in the art that falls within the categories of acrylic and vinyl monomers (including monomers modified with a silicone chain).

As other vinyl monomers that may be used, mention may also be made of:

N-vinylpyrrolidone, vinylcaprolactam, vinyl-N-(C₁-C₆)alkylpyrroles, vinyloxazoles, vinylthiazoles, vinylpyrimidines and vinylimidazoles,

olefins such as ethylene, propylene, butylene, isoprene and butadiene.

The vinyl polymer may be partially crosslinked with one or more difunctional monomers, especially comprising at least two ethylenic unsaturations, such as ethylene glycol dimethacrylate or diallyl phthalate.

In a non-limiting manner, the polymers in dispersion of the invention may be chosen from the following polymers or copolymers: polyurethanes, polyurethane-acrylics, polyureas, polyurea-polyurethanes, polyester-polyurethanes, polyether-polyurethanes, polyesters, polyesteramides, fatty-chain polyesters, alkyds; acrylic and/or vinyl polymers or copolymers; acrylic-silicone copolymers; polyacrylamides; silicone polymers, for instance silicone polyurethanes or silicone acrylics, and fluoro polymers and mixtures thereof. The polymer(s) in oily dispersion may represent (as solids or active material) from 0.1% to 60% of the weight of the composition, preferably from 2% to 40% and better still from 4% to 25%. For a stabilizer that is solid at ambient temperature, the amount of solids in the dispersion represents the total amount of polymer and stabilizer.

The liposoluble or dispersible polymers in the composition of the invention can be also used in an amount of from 0.01% to 20% (as active material) relative to the total weight of the composition, such as, for example, from 1% to 10%, if they are present.

III) Aqueous Dispersions of Polymer Particles

According to another embodiment, the at least one film-forming polymer can be chosen from aqueous dispersions of polymer particles, in case the composition according to the invention comprises an aqueous phase.

The aqueous dispersion comprising one or more film-forming polymers may be prepared by the person skilled in the art on the basis of his general knowledge, in particular by emulsion polymerization or by placement in dispersion of the previously formed polymer.

Among the film-forming polymers which may be used in the composition according to this invention, there may be cited synthetic polymers of the polycondensate type or the radical type, polymers of natural origin, and mixtures thereof.

Among the polycondensates, there also may be cited the anionic, cationic, nonionic or amphoteric polyurethanes, polyurethane-acrylics, polyurethane-polyvinylpyrrolidones, polyester-polyurethanes, polyether-polyurethanes, polyureas, polyurea/polyurethanes, and mixtures thereof.

The polyurethanes may be, for example, an aliphatic, cycloaliphatic or aromatic polyurethane, polyurea/urethane or polyurea copolymer comprising, alone or as a mixture:

at least one sequence of linear or branched aliphatic and/or cycloaliphatc and/or aromatic polyester origin, and/or

at least one sequence of aliphatic and/or cycloaliphatc and/or aromatic polyether origin, and/or

at least one silicone sequence, substituted or unsubstituted, branched or unbranched, for example polydimethylsiloxane or polymethylphenylsiloxane, and/or

at least one sequence comprising fluorous groups.

The polyurethanes such as defined in the invention also may be obtained from branched or unbranched polyesters or from alkyds comprising mobile hydrogens which are modified by means of polyaddition with a diisocyanate and an organic bifunctional co-reactive (for example dihydro, diamino or hydroxyamino) compound, further comprising either a carboxylate or carboxylic acid group, or a sulfonic acid or sulfonate group, or even a neutralizable tertiary amine group or a quaternary ammonium group.

There also may be cited the polyesters, polyester amides, polyesters with a fatty chain, polyamides, and epoxyester resins.

The polyesters may be obtained in known manner by means of polycondensation of aliphatic or aromatic diacids with aliphatic or aromatic diols or with polyols. Succinic acid, glutaric acid, adipic acid, pimelic acid, suberic acid or sebacic acid may be used as aliphatic diacids. Terephthalic acid or isophthalic acid, or else even a derivative such as phthalic anhydride, may be used as aromatic diacids. Ethylene glycol, propylene glycol, diethylene glycol, neopentyl glycol, cyclohexane dimethanol, 4,4′-(1-methylpropylidene)bisphenol, may be used as aliphatic diols. Glycerol, pentaerythritol, sorbitol, trimethylol propane may be used as polyols.

The polyester amides may be obtained in a manner analogous to the polyesters, by means of polycondensation of diacids with diamines or amino alcohols. Ethylene diamine, hexamethylenediamine, meta- or paraphenylene diamine may be used as diamine. Monoethanolamine may be used as amino alcohol.

As a monomer bearing an anionic group which may be used during polycondensation, there may be cited, for example, dimethylol propionic acid, trimellitic acid or a derivative such as trimellitic anhydride, the sodium salt of 3-sulfo pentanediol acid, the sodium salt of 5-sulfo 1,3-benzenedicarboxylic acid. Polyesters with a fatty chain may be obtained through the use of diols with a fatty chain during polycondensation. The epoxyester resins may be obtained by polycondensation of fatty acids with a condensate at the α,ω-diepoxy ends.

The radical-type polymers may be in particular acrylic and/or vinyl polymers or copolymers. Anionic radical polymers preferably are used. As a monomer bearing an anionic group which may be used during radical polymerization, there may be cited acrylic acid, methacrylic acid, crotonic acid, maleic anhydride, 2-acrylamido 2-methyl propane sulfonic acid.

The acrylic polymers may result from the copolymerization of monomers chosen from among the esters and/or amides of acrylic acid or methacrylic acid. As examples of ester-type monomers, there may be cited methyl methacrylate, ethyl methacrylate, butyl methacrylate, isobutyl methacrylate, 2-ethyl hexyl methacrylate, lauryl methacrylate. As examples of amide-type monomers, there may be cited N-t-butyl acrylamide and N-t-octyl acrylamide.

There preferably are used acrylic polymers obtained by copolymerization of monomers with ethylene unsaturation containing hydrophilic groups, preferably of a nonionic nature, such as hydroxyethyl acrylate, 2-hydroxypropyl acrylate, hydroxyethyl methacrylate, 2-hydroxypropyl methacrylate.

The vinyl polymers may result from the homopolymerization or from the copolymerization of monomers chosen from among the vinyl esters, styrene, or butadiene. As examples of vinyl esters, there may be cited vinyl acetate, vinyl neodecanoate, vinyl pivalate, vinyl benzoate and vinyl t-butyl benzoate.

Acrylic/silicone copolymers or even nitrocellulose/acrylic copolymers also may be used.

Polymers of natural origin, possibly modified, may be chosen from among shellac resin, sandarac gum, the dammars, elemis, copals, cellulosic derivatives, and mixtures thereof.

There also may be cited the polymers resulting from the radical polymerization of one or more radical monomers, inside and/or partially on the surface, of preexisting particles of at least one polymer chosen from among the group consisting of polyurethanes, polyureas, polyesters, polyester amides and/or alkyds. These polymers generally are called hybrid polymers.

When an aqueous dispersion of polymer particles is used, the dry-matter content of said aqueous dispersion may be on the order of 5-60% by weight, and preferably 30-50%.

The size of the polymer particles in aqueous dispersion may range between 10-500 nm, and preferably ranges between 20 and 150 nm, which makes it possible to obtain a film having a notable sheen. Particle sizes ranging up to one micron, however, may be used.

IV Water-Soluble Polymers

According to one embodiment according to the invention, the film-forming polymer may be a water-soluble polymer and is therefore present in the aqueous phase of the composition in solubilized form. The composition according to the invention is not anhydrous in that case. Among the water-soluble film-forming polymers, the following cationic polymers may be cited:

(1) acrylic polymers or copolymers such as polyacrylates or polymethacrylates; The copolymers of family (1) furthermore may contain one or more moieties deriving from comonomers which may be chosen from within the family of acrylamides, methacrylamides, diacetone acrylamides, acrylamides and methacrylamides substituted on nitrogen with lower alkyls, acrylic or methacrylic acids or esters thereof, vinylactams such as vinylpyrrolidone or vinylcaprolactam, vinyl esters.

Thus, among these copolymers of family (1), there may be cited:

the copolymers of acrylamide and dimethylaminoethyl methacrylate quaternized with dimethyl sulfate, or with a dimethyl halogenide such as that sold under the name HERCOFLOC by the company HERCULES,

the copolymers of acrylamide and methacryloyloxyethyltrimethylammonium chloride described, for example, in patent application EP-A-080976 and sold under the name BINA QUAT P 100 by the company CIBA GEIGY,

the copolymer of acrylamide and methacryloyloxyethyltrimethylammonium methosulfate sold under the name RETEN by the company HERCULES,

the vinylpyrrolidone/dialkylaminoalkyl acrylate or methacrylate copolymers, quaternized or non-quaternized, such as the products sold under the name “GAFQUAT” by the company ISP such as, for example, “GAFQUAT 734” or “GAFQUAT 755” or else the products designated as “COPOLYMER 845, 958 and 937.” These polymers are described in detail in French patents 2.077.143 and 2.393.573,

the dimethylaminoethyl methacrylate/vinylcaprolactam/vinylpyrrolidone terpolymers such as the product sold under the name GAFFIX VC 713 by the company ISP, and

the quaternized vinylpyrrolidone/dimethylaminopropyl methacrylamide copolymer such as the product sold under the name “GAFQUAT HS 100” by the company ISP.

(2) the quaternized polysaccharides described most especially in patents U.S. Pat. No. 3,589,578 and U.S. Pat. No. 4,031,307, such as the guar gums containing cationic trialkylammonium groups. Such products are marketed in particular under the trade names of JAGUAR C13 S, JAGUAR C 15, JAGUAR C 17 by the company MEYHALL.

(3) the quaternary vinylpyrrolidone and vinylimidazole copolymers;

(4) the chitosans or salts thereof;

(5) the cationic cellulose derivatives such as copolymers of cellulose or cellulose derivatives grafted with a water-soluble monomer comprising a quaternary ammonium and described in particular in the patent U.S. Pat. No. 4,131,576, such as the hydroxyalkyl celluloses, like the hydroxymethyl, hydroxyethyl or hydroxypropyl celluloses grafted in particular with a methacryloyloxyethyl trimethylammonium, methacrylamidopropyl trimethylammonium, dimethyl-diallylammonium salt. The marketed products corresponding to this definition are most especially the products sold under the name “CELQUAT L200” and “CELQUAT H 100” by the National Starch Company.

Among the film-forming water-soluble polymers, the following amphoteric polymers may be cited:

(1) the polymers resulting from the copolymerization of a monomer derived from a vinyl compound bearing a carboxylic group such as most especially acrylic acid, methacrylic acid, maleic acid, alpha-chloracrylic acid, and of a basic monomer derived from a substituted vinyl compound containing at least one base atom such as most particularly dialkylaminoalkylmethacrylate and acrylate, dialkylaminoalkylmethacrylamide and acrylamide. Such compounds are described in the American patent U.S. Pat. No. 3,836,537.

(2) the polymers comprising moieties deriving:

a) from at least one monomer chosen from among the acrylamides or methacrylamides substituted on nitrogen with an alkyl radical,

b) from at least one acid comonomer containing one or more reactive carboxylic groups, and

c) from at least one basic comonomer such as esters, with primary, secondary, tertiary and quaternary amine substituents, of acrylic and methacrylic acids, and the product of quaternization of dimethylaminoethyl methacrylate with dimethyl or diethyl sulfate.

(3) the crosslinked alkoyl polyamino amides deriving in whole or in part from polyaminoamides

(4) the polymers comprising zwitterionic moieties

(5) the polymer derived from chitosan

(6) the polymers derived from N-carboxyalkylation of chitosan, such as N-carboxymethyl chitosan or N-carboxybutyl chitosan sold under the name “EVALSAN” by the company JAN DEKKER.

(7) the copolymers of alkyl(C1-C5)vinylether/maleic anhydride partially modified by semiamidification with an N,N-dialkylaminoalkylamine such as N,N-dimethylaminopropylamine or by semiesterification with an N,N-dialcanolamine, These copolymers also may comprise other vinyl comonomers such as vinylcaprolactam.

Water-soluble film-forming polymers are preferably selected in the group consisting of:

proteins such as proteins of plant origin, such as wheat or soya bean proteins; proteins of animal origin such as keratin, for example keratin hydrolysates and sulphonic keratins;

anionic, cationic, amphoteric or nonionic polymers of chitin or chitosan;

cellulose polymers such as hydroxyethyl cellulose, hydroxypropyl cellulose, methyl cellulose, ethyl hydroxyethyl cellulose, carboxymethyl cellulose, and quaternized derivatives of cellulose;

acrylic polymers or copolymers such as polyacrylates or polymethacrylates;

vinyl polymers, such as polyvinylpyrrolidones, copolymers of methyl vinyl ether and maleic anhydride, the copolymer of vinyl acetate and crotonic acid, copolymers of vinylpyrrolidone and vinyl acetate;

copolymers of vinylpyrrolidone and caprolactam; polyvinyl alcohols;

optionally modified polymers of natural origin, such as:

gum arabic, guar gum, xanthan derivatives, karaya gum;

alginates and carrageenans;

glycoaminoglycans, hyaluronic acid and its derivatives;

shellac resin, sandarac gum, dammars, elemis, copals;

deoxyribonudeic acid;

muccopolysaccharides such as hyaluronic acid, chondroitin sulphate, and mixtures thereof.

These polymers will be used in particular if a more or less appreciable elimination of the film with water is desired.

In order to improve the film-forming nature of an oily or aqueous polymer, it is possible to add to the polymeric system a coalescent agent which may be chosen from among the known coalescent agents.

According to a preferred embodiment of the invention, the film-forming polymer is chosen from among the polymers with a non-silicone organic backbone grafted with monomers containing a polysiloxane. These polymers may be liposoluble, lipodispersible, water-soluble or dispersible in an aqueous medium, as the case may be.

The polymers with a non-silicone organic backbone grafted with monomers containing a polysiloxane consist of a main organic chain formed from organic monomers not comprising silicone, on which there is grafted, within said chain as well as possibly on at least one of the ends thereof, at least one polysiloxane macromer.

In the following, “polysiloxane macromer” is understood to denote, as generally accepted, any monomer containing a polysiloxane-type polymer chain in its structure.

The non-silicone organic monomers making up the main chain of the grafted silicone polymer may be chosen from among the monomers with ethylene unsaturation which are polymerizable by the radical method, monomers polymerizable by polycondensation such as those forming polyamides, polyesters, polyurethanes, cycle-opening monomers such as those of the oxazoline or caprolactone type.

The polymers with a non-silicone organic backbone grafted with monomers containing a polysiloxane according to this invention may be obtained in accordance with any method known to the person skilled in the art, in particular by reaction between (i) a starting polysiloxane macromer correctly functionalized on the polysiloxane chain and (ii) and one or more non-silicone organic compounds, themselves correctly functionalized with a function which is capable of reacting with the functional group or groups borne by said silicone by forming a covalent bond; a classic example of such a reaction is the radical reaction between a vinyl group borne on one of the ends of the silicone with a double bond of a monomer with ethylene unsaturation of the main chain.

The polymers with a non-silicone organic backbone grafted with monomers containing a polysiloxane according to the invention preferably are chosen from among those described in the patents U.S. Pat. Nos. 4,693,935, 4,728,571 and 4,972,037 and patent applications EP-A-0 412704, EP-A-0 412707, EP-A-0 640 105 and WO 95/00578. It concerns copolymers obtained by radical polymerization starting from monomers with ethylene unsaturation and silicone monomers having a vinyl end group or else copolymers obtained by reaction of a polyolefin containing functionalized groups and a polysiloxane macromer having an end function reactive with said functionalized groups.

A special family of grafted silicone polymers suitable for the implementation of this invention consists of the grafted silicone polymers containing:

a) from 0 to 98% by weight of at least one lipophilic monomer (A) of low lipophilic polarity with ethylene unsaturation, polymerizable by the radical method;

b) from 0 to 98% by weight of at least one hydrophilic polar monomer (B) with ethylene unsaturation, copolymerizable with the monomer or monomers of type (A);

c) from 0.01 to 50% by weight of at least one polysiloxane macromer (C) of general formula:

X(Y)ₙSi(R)₃₋ₘZₘ (I)

in which:

X designates a vinyl group copolymerizable with the monomers (A) and (B);

Y designates a group with a divalent bond;

R designates a hydrogen, a C₁-C₆ alkyl or alkoxy, a C₆-C₁₂ aryl;

Z designates a monovalent polysiloxane moiety having an average molecular weight by number of at least 500;

n is 0 or 1 and m is an integer ranging from 1 to 3; the percentages being calculated in relation to the total weight of the monomers (A), (B) and (C).

These polymers have an average molecular weight by number ranging from 10,000 to 2,000,000 and preferably a glass transition temperature Tg or a crystalline melting temperature Tm of at least −20° C.

As examples of lipophilic monomers (A) there may be cited C₁-C₁₈ alcohol esters of acrylic or methacrylic acid; styrene; polystyrene macromers; vinyl acetate; vinyl propionate; alpha-methylstyrene; tertiobutylstyrene; butadiene; cyclohexadiene; cyclohexadiene [sic]; ethylene; propylene; vinyltoluene; esters of acrylic or methacrylic acid and of 1,1-dyhydroperfluoroalkanols or homologues thereof; esters of acrylic or methacrylic acid and omega-hydrydofluoralkanols; esters of acrylic or methacrylic acid and fluoroalkylsulfoamido-alcohols; esters of acrylic or methacrylic acid and fluoroalkyl alcohols; esters of acrylic or methacrylic acid and alcohol fluoroethers; or mixtures thereof. The preferred monomers (A) are chosen from within the group consisting of n-butyl methacrylate, isobutyl methacrylate, tertio-butyl acrylate, tertio-butyl methacrylate, 2-ethylhexyl methacrylate, methyl methacrylate, 2-(N-methyl perfluoro-octane sulfonamido)-ethylacrylate; 2-(N-butylperfluorooctane sulfonamido)-ethyl-acrylate or mixtures thereof.

As examples of polar monomers (B) there may be cited acrylic acid, methacrylic acid, N,N-dimethylacrylamide, dimethylaminoethyl methacrylate, quaternized dimethylaminoethyl methacrylate, (meth)acrylamide, N-t-butyl acrylamide, maleic acid, maleic anhydride and demi-esters thereof, hydroxyalkyl (meth)acrylates, diallyldimethylammonium chloride, vinyl-pyrrolidone, vinyl ethers, maleimides, vinylpyridine, vinylimidazole, heterocyclic vinyl polar compounds, styrene sulfonate, allyl alcohol, vinyl alcohol, vinyl caprolactam or mixtures thereof. The monomers (B) preferably are chosen from within the group consisting of acrylic acid, N,N-dimethylacrylamide, dimethylaminoethyl methacrylate, quaternized dimethylaminoethyl methacrylate, vinylpyrrolidone and mixtures thereof.

The polysiloxane macromers (C) of formula (I) preferably are chosen from among those corresponding to the following general formula (II):

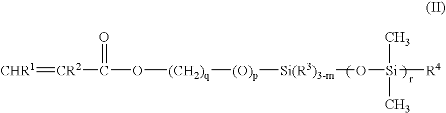

in which:

R¹ is hydrogen or —COOH (preferably hydrogen);

R² is hydrogen, methyl or —CH₂COOH (preferably methyl);

R³ is C₁-C₆ alkyl, alkoxy or alkylamino, C₆-C₁₂ aryl or hydroxyl (preferably methyl);

R⁴ is C₁-C₆ alkyl, alkoxy or alkylamino, C₆-C₁₂ aryl or hydroxyl (preferably methyl);

q is an integer from 2 to 6 (preferably 3);

p is 0 or 1;

r is a whole number from 5 to 700;

m is an integer ranging from 1 to 3 (preferably 1);

There preferably are used polysiloxane macromers of formula:

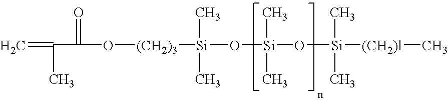

n being a number ranging from 5 to 700 and I being an integer ranging between 0 and 3.

One embodiment of the invention consists in using a copolymer able to be obtained by radical polymerization starting from the mixture of monomers consisting of:

a) 60% by weight of tertiobutyl acrylate;

b) 20% by weight of acrylic acid;

c) 20% by weight of silicone macromer of formula:

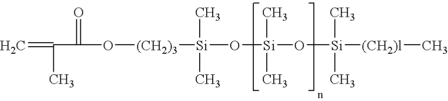

with n being a number ranging from 5 to 700 and I being an integer ranging between 0 and 3, the percentages by weight being calculated in relation to the total weight of the monomers.

Another special embodiment of the invention consists in using a copolymer able to be obtained by radical polymerization starting from the mixture of monomers consisting of:

a) 80% by weight of tertiobutyl acrylate;

b) 20% by weight of silicone macromer of formula:

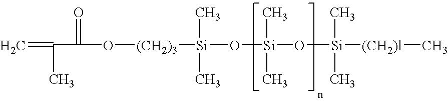

with n being a number ranging from 5 to 700 and I being an integer ranging between 0 and 3, the percentages by weight being calculated in relation to the total weight of the monomers.

Another special family of grafted silicone polymers with a non-silicone organic backbone suitable for implementation of this invention consists of the grafted silicone copolymers able to be obtained by reactive extrusion of a polysiloxane macromer having an end reactive function on a polymer of the polyolefin type comprising reactive groups capable of reacting with the end function of the polysiloxane macromer to form a covalent bond allowing for the grafting of the silicone on the main chain of the polyolefin. These polymers, as well as the process for preparation thereof, are described in patent application WO 95/00578.

The reactive polyolefins preferably are chosen from among the polyethylenes or the polymers of monomers derived from ethylene such as propylene, styrene, alkyl styrene, butylene, butadiene, the (meth)acrylates, the vinyl esters or equivalents, comprising reactive functions capable of reacting with the end function of the polysiloxane macromer. They are chosen most particularly from among the copolymers of ethylene or ethylene derivatives and monomers chosen from among those comprising a carboxylic function such as (meth)acrylic acid; those comprising an acid anhydride function such as maleic acid anhydride; those comprising an acid chloride function such as (meth)acrylic acid chloride; those comprising an ester function such as the esters of (meth)acrylic acid; those comprising an isocyanate function.

The silicone macromers preferably are chosen from among the polysiloxanes comprising a functionalized group, at the end of the polysiloxane chain or near the end of said chain, chosen from within the group consisting of alcohols, thiols, epoxy, primary and secondary amines, and most particularly from among those corresponding to the general formula:

T—(CH₂)ₛ—Si—[—(OSiR⁵R⁶)ₜ—R⁷]^{y} (III)

in which T is chosen from within the group consisting of NH2, NHR′, an epoxy function, OH, SH; R⁵, R⁶, R⁷ and R′, independently, denote a C₁-C₆ alkyl, a C₆-C₁₂ phenyl, benzyl or alkylphenyl, hydrogen; s is a number from 2 to 00 [sic], t is a number from 0 to 1000 and y is a number from 1 to 3. They have an average molecular weight by number preferably ranging from 5,000 to 300,000, more preferably from 8,000 to 200,000 and more particularly from 9,000 to 40,000.

According to a preferred embodiment, the film-forming polymer may be purchased from Minnesota Mining and Manufacturing Company under the trade names “Silicone Plus” polymers. For example, poly(isobutyl methacrylate-co-methyl FOSEA)-g-poly(dimethylsiloxane) is sold under the trade name SA 70-5 IBMMF.

According to another preferred form of this invention, the film-forming polymer is chosen from among the silicone polymers grafted with non-silicone organic monomers. These polymers may be liposoluble, lipodispersible, water-soluble or dispersible in an aqueous medium, as the case may.

Said grafted silicone polymer or polymers, with a polysiloxane backbone grafted with non-silicone organic monomers containing a main silicone (or polysiloxane (≡Si—O—)ₙ) chain on which there is grafted, within said chain as well as possibly on at least one of its ends, at least one organic group not comprising silicone.

The polymers with a polysiloxane backbone grafted with non-silicone organic monomers according to the invention may be existing commercial products or else obtained according to any means known to the person skilled in the art, in particular by reaction between (i) a starting silicone correctly functionalized on one or more of these silicon atoms and (ii) a non-silicone organic compound itself correctly functionalized with a function which is capable of reacting with the functional group or groups borne by said silicone by forming a covalent bond; a classic example of such a reaction is the hydroxsylilation reaction between ≡Si—H groups and CH₂═CH— vinyl groups, or else the reaction between —SH thio-functional groups with these same vinyl groups.

Examples of polymers with a polysiloxane backbone grafted with non-silicone organic monomers suitable for implementation of this invention, as well as the specific method of preparation thereof, are described in particular in patent applications EP-A-0 582 152, WO 93/23009 and WO 95/03776, the teachings of which are included in full in this description by way of non-restrictive references.

According to a particularly preferred embodiment of this invention, the silicone polymer, with a polysiloxane backbone grafted with non-silicone organic monomers which is utilized is composed of the result of radical copolymerization between, on the one hand, at least one non-silicone anionic organic monomer having an ethylene unsaturation and/or a non-silicone hydrophobic organic monomer having an ethylene unsaturation and, on the other, a silicone having in its chain at least one, and preferably several, functional groups capable of reacting on said ethylene unsaturations of said non-silicone monomers by forming a covalent bond, in particular thio-functional groups.

According to this invention, said anionic monomers with ethylene unsaturation preferably are chosen, alone or as a mixture, from among the linear or branched unsaturated carboxylic acids, possibly neutralized in whole or in part in the form of a salt, this or these unsaturated carboxylic acid(s) being able to be most particularly acrylic acid, methacrylic acid, maleic acid, maleic anhydride, itaconic acid, fumaric acid and crotonic acid. The suitable salts are in particular alkaline, alkaline-earth and ammonium salts. It likewise will be noted that, in the final grafted silicone polymer, the organic group with an anionic nature which is composed of the result of radical (homo)polymerization of at least one unsaturated carboxylic-acid-type anionic monomer may be, after reaction, post-neutralized with a base (soda, ammonia, . . . ) to bring it to the form of a salt.

According to this invention, the hydrophobic monomers with ethylene unsaturation preferably are chosen, alone or as a mixture, from among the alkanol acrylic acid esters and/or the alkanol methacrylic acid esters. The alkanols preferably are C₁-C₁₈ and more particularly C₁-C₁₂. The preferred monomers are chosen from within the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tertio-butyl (meth)acrylate, tridecyl (meth)acrylate, stearyl (meth)acrylate or mixtures thereof.

A family of silicone polymers with a polysiloxane backbone grafted with non-silicone organic monomers particularly well suited for implementation of this invention consists of silicone polymers comprising in their structure the moiety of formula IV below:

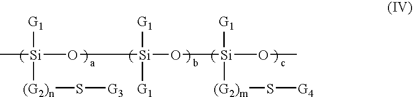

in which the radicals G₁, identical or different, represent hydrogen or a C₁-C₁₀ alkyl radical or even a phenyl radical; the radicals G₂, identical or different, represent represents [sic] a C₁-C₁₀ alkylene group; G₃ represents a polymeric residue resulting from (homo)polymerization of at least one anionic monomer with ethylene unsaturation; G₄ represents a polymeric residue resulting from (homo)polymerization of at least one monomer of at least one hydrophobic monomer [sic] with ethylene unsaturation; m and n are equal to 0 or 1; a is a whole number ranging from 0 to 50; b is a whole number which may range between 10 and 350, c is a whole number ranging from 0 to 50; on condition that one of the parameters a and c is other than 0.

The moiety of formula (IV) hereinabove preferably has at least one, and more preferably still, all of the following characteristics:

the G₁ radicals denote an alkyl radical, preferably the methyl radical;

n is not zero, and the G₂ radicals represent a divalent C₁-C₃ radical, preferably a propylene radical;

G₃ represents a polymeric radical resulting from (homo)polymerization of at least one monomer of the carboxylic acid type with ethylene unsaturation, preferably acrylic acid and/or methacrylic acid;

G₄ represents a polymeric radical resulting from (homo) polymerization of at least one monomer of the C₁-C₁₀ alkyl (meth)acrylate type, preferably isobutyl or methyl (meth)acrylate.

Examples of silicone polymers corresponding to the formula (IV) are in particular polydimethylsiloxanes (PDMS) on which there are grafted, through a thiopropylene-type secondary linking, mixed polymer moieties of the poly(meth)acrylic acid type and of the alkyl poly(meth)acrylate type.

Other examples of silicone polymers corresponding to formula (IV) are in particular polydimethylsiloxanes (PDMS) on which there are grafted, through a thiopropylene-type secondary linking, polymer moieties of the isobutyl poly(meth)acrylate type.

Such polymers include polymers comprising at least one group of the formula:

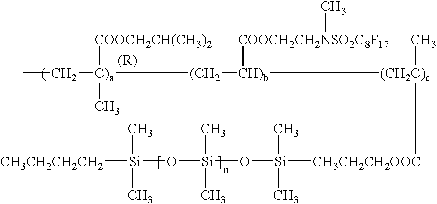

wherein

a, b, and c, which may be identical or different, are each a number ranging from 1 to 100,000; and the terminal groups, which may be identical or different, are each chosen from C₁-C₂₀ linear alkyl groups, C₃-C₂₀ branched chain alkyl groups, C₃-C₂₀ aryl groups, C₁-C₂₀ linear alkoxy groups, and C₃-C₂₀ branched alkoxy groups.

Such polymers are disclosed in U.S. Pat. Nos. 4,972,037, 5,061,481, 5,209,924, 5,849,275, and 6,033,650, and WO 93/23446 and WO 95/06078.

Another family of silicone polymers with a polysiloxane backbone grafted with non-silicone organic monomers particularly well suited to implementation of this invention consists of the silicone polymers comprising in their structure the moiety of formula (V) below

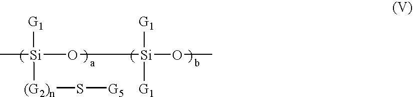

in which the radicals G₁ and G₂ have the same meaning as before; G₅ represents a polymeric residue resulting from (homo)polymerization of at least one monomer of at least one hydrophobic monomer [sic] with ethylene unsaturation or copolymerization of at least one anionic monomer with ethylene unsaturation and at least one hydrophobic monomer with ethylene unsaturation; n is equal to 0 or 1; a is a whole number ranging from 0 to 50; b is a whole number which may range between 10 and 350; on condition that a is other than 0.

The moiety of formula (V) hereinabove preferably has at least one, and more preferably still all, of the following characteristics:

the radicals G₁ denote an alkyl radical, preferably the methyl radical;

n is not zero, and the radicals G₂ represent a C₁-C₃ divalent radical, preferably a propylene radical.

The molecular mass by number of the silicone polymers with a polysiloxane backbone grafted with non-silicone organic monomers of the invention preferably varies from approximately 10,000 to 1,000,000, and more preferably still from approximately 10,000 to 100,000.

The composition may contain from 2-60% by weight, better from 5 to 60%, preferably from 2-30% by weight of film-forming-polymer dry matter. More generally, the total quantity of polymer should be in sufficient quantity to form on the skin and/or the lips a cohesive film which is able to follow the movements of the skin and/or the lips without peeling away or cracking.

When the polymer has a glass transition temperature that is too high for the desired use, a plasticizer may be combined therewith so as to lower this temperature of the mixture used. The plasticizer may be chosen from the plasticizers usually used in the field of application, and especially from compounds which may be solvents for the polymer.

The composition also may contain at least one hydrophilic or hydrophobic plasticizing agent, chosen for its compatibility with the polymer or polymers and in a quantity such that it does not impair the sensitivity of the film to water. Said plasticizing agent may be chosen from among all the compounds known to the person skilled in the art as being capable of fulfilling the sought function. This agent may be water soluble or insoluble in water and possibly may exist in the form of an aqueous dispersion.

There may be cited in particular, alone or as a mixture, the usual plasticizers such as:

glycols and derivatives thereof such as diethylene glycol ethylether, diethylene glycol methylether, diethylene glycol butylether or even diethylene glycol hexylether, ethylene glycol ethylether, ethylene glycol butylether, ethylene glycol hexylether,

esters of glycerol,

the derivatives of propylene glycol and in particular propylene glycol phenylether, propylene glycol diacetate, dipropylene glycol butylether, tripropylene glycol butylether, propylene glycol methylether, dipropylene glycol ethylether, tripropylene glycol methylether and diethylene glycol methylether, propylene glycol butylether,

esters of acids, in particular carboxylic, such as citrates, phthalates, adipates, carbonates, tartrates, phosphates, sebaceates,

oxyethylene derivatives such as oxyethylene oils, in particular vegetable oils such as castor oil; silicone oils.

The quantity of plasticizing agent may be chosen by the person skilled in the art on the basis of his general knowledge, in such manner as to obtain a film having the desired mechanical properties, while preserving the composition of the cosmetically acceptable properties.

According to the invention, the system comprising the film-forming polymer or polymers, the possible coalescent agents and the possible plasticizers shall be called “polymeric system”; this polymeric system is to be capable of forming a film on the support on which it is deposited, supple, flexible, cohesive, following the movements of the support (lips or skin) on which it is deposited.

When the composition is in the form of a foundation or a lipstick, the film obtained with said composition preferably shall bear out, in the measurement conditions defined preceding the examples, at least one of the following physico-chemical conditions:

A Young modulus less than approximately 200 MPa, preferably less than approximately 100 MPa, and preferred less than 80 MPa, and/or

an elongation in excess of approximately 200% and, preferably, in excess of 300%, and/or

a hardness less than 110, preferably less than 70, more preferably less than 55.

Additional Additives

The composition of the invention can also comprise any additive usually used in the field under consideration, chosen in particular from dispersants such as poly(2-hydroxystearic acid), antioxidants, essential oils, preserving agents, fragrances, waxes, fillers, neutralizing agents, cosmetic and dermatological active agents such as, for example, emollients, moisturizers, vitamins, essential fatty acids, sunscreens, and mixtures thereof. These additives may be present in the composition in a proportion of from 0% to 20% (such as from 0.01% to 20%) relative to the total weight of the composition and further such as from 0.01% to 10% (If present).

The composition of the invention can also contain, as an additive, an aqueous phase containing water that is optionally thickened or gelled with an aqueous-phase thickener or gelling agent and/or containing ingredients soluble in water. The water can represents from 0.01 to 50%, for example from 0.5 to 30% relative to the total weight of the composition.

Needless to say, a person skilled in the art will take care to select the optional additional additives and/or the amount thereof such that the advantageous properties of the composition according to the invention are not, or are not substantially, adversely affected by the envisaged addition.

The composition according to the invention can be in the form of a tinted or non tinted dermatological composition or a care composition for keratin materials such as the skin, the lips and/or superficial body growths, in the form of an antisun composition or body hygiene composition in particular in the form of a deodorant product or make-up-removing product in stick form. It can be used in particular as a care base for the skin, superficial body growths or the lips (lip balms, for protecting the lips against cold and/or sunlight and/or the wind, or care cream for the skin, the nails or the hair). As defined herein, a deodorant product is personal hygiene product and does not relate to care, make-up or treatment of keratin materials, including keratinous fibers.

The composition of the invention may also be in the form of a colored make-up product for the skin, in particular a foundation, optionally having care or treating properties, a blusher, a face powder, an eye shadow, a concealer product, an eyeliner, a make-up product for the body; a make-up product for the lips such as a lipstick, optionally having care or treating properties; a make-up product for superficial body growths such as the nails or the eyelashes, in particular in the form of a mascara cake, or for the eyebrows and the hair, in particular in the form of a pencil.

Needless to say, the composition of the invention should be cosmetically or dermatologically acceptable, i.e. it should contain a non-toxic physiologically acceptable medium and should be able to be applied to the skin, superficial body growths or the lips of human beings. For the purposes of the invention, the expression “cosmetically acceptable” means a composition of pleasant appearance, odor, feel and taste.

The composition advantageously contains at least one cosmetic active agent and/or at least one dermatological active agent, i.e., an agent having a beneficial effect on the skin, lips or body growths and/or at least one coloring agent.

Coloring Agents

The coloring agent according to the invention may be chosen from the lipophilic dyes, hydrophilic dyes, pigments and nacreous pigments (i.e., nacres) usually used in cosmetic or dermatological compositions, and mixtures thereof. This coloring agent is generally present in a proportion of from 0.01% to 50% relative to the total weight of the composition, such as from 0.5% to 40% and further such as from 5% to 30%, if it is present. In the case of a composition in the form of a free or compacted powder, the amount of coloring agent in the form of solid particles that are insoluble in the medium (nacres and/or pigments) may be up to 90% relative to the total weight of the composition.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow or annatto. They can represent from 0.1% to 20% of the weight of the composition, for example, from 0.1% to 6% (if present). The water-soluble dyes are, for example, beetroot juice or methylene blue, and can represent up to 6% of the total weight of the composition.

The pigments may be white or colored, goniochromatic or not, mineral and/or organic, and coated or uncoated. Among the mineral pigments which may be mentioned are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments that may be mentioned are carbon black, pigments of D&C type, and lakes based on cochineal carmine or on barium, strontium, calcium or aluminium. The pigments can represent from 0.1% to 50%, such as from 0.5% to 40% and further such as from 2% to 30% relative to the total weight of the composition, if they are present.

The nacreous pigments may be chosen from white nacreous pigments such as mica coated with titanium or with bismuth oxychloride, colored nacreous pigments such as titanium mica with iron oxides, titanium mica with, in particular, ferric blue or chromium oxide, titanium mica with an organic pigment of the type mentioned above, as well as nacreous pigments based on bismuth oxychloride. They can represent, for example, from 0.1% to 20% relative to the total weight of the composition, and further such as from 0.1% to 15%, if they are present.

In one embodiment, the coloring agent is a pigment (nacreous or not).

In another embodiment, the pigment is treated or not treated, and is preferably hydrophobic.

Waxes

The composition can optionally contain one or more waxes to improve the structuring in stick form, although this rigid form can be obtained in the absence of wax. For the purposes of the present invention, a wax is a lipophilic fatty compound that is solid at room temperature (25° C.) and atmospheric pressure (760 mmHg, i.e. 101 KPa), which undergoes a reversible solid/liquid change of state, having a melting point of greater than 40° C. and further such as greater than 55° C. and which may be up to 200° C. and having an anisotropic crystal organization in the solid state. The size of the crystals is such that the crystals diffract and/or scatter light, giving the composition a cloudy, more or less opaque appearance. By bringing the wax to its melting point, it is possible to make it miscible with oils and to form a microscopically homogeneous mixture, but on returning the temperature of the mixture to room temperature, recrystallization of the wax in the oils of the mixture is obtained. It is this recrystallization in the mixture which is responsible for the reduction in the gloss of the mixture. Thus, the composition advantageously contains little or no wax, and in particular less than 5% wax.

For the purposes of the invention, the waxes are those generally used in cosmetics and dermatology; they are, for example, of natural origin, for instance beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C. for example, at above 55° C. silicone waxes such as alkyl- and alkoxy-poly(di)methylsiloxanes and/or poly(di)methyl-siloxane esters that are solid at 40° C. for example, at above 55° C.

According to the invention, the melting point values correspond to the melting peak measured by the “Differential Scanning Calorimetry” method with a temperature rise of 5 or 10° C./min.

The composition according to the invention may be manufactured by the known processes that are generally used in cosmetics or dermatology. It may be manufactured by the process which comprises heating the polymer at least to its softening point, adding the film-forming agent(s), the coloring agent(s) and the additive(s) thereto and then mixing everything together until a clear, transparent solution is obtained. After reducing the temperature, the volatile solvent(s) is(are) then added to the mixture obtained. The homogeneous mixture obtained can then be cast in a suitable mould such as a lipstick mould or directly into the packaging articles (case or dish in particular).

Another aspect of the invention is a lipstick composition in stick form at least one liquid fatty phase comprising (i) at least one oil structured with at least one structuring polymer consisting of a polymer (homopolymer or copolymer) with a weight-average molecular mass ranging from 500 to 500,000, containing at least one moiety comprising:

at least one polyorganosiloxane group, consisting of from 1 to 1,000 organosiloxane units in the chain of the moiety or in the form of a graft, and

at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,

the polymer being solid at room temperature and soluble in said oil at a temperature of from 25 to 250° C. and

(ii) at least one film-forming agent dispersed or solubilized in the liquid fatty phase,

said oil having an affinity with said structuring polymer and optionally with the film-forming agent, and

the liquid fatty phase, the polymer and the film-forming agent forming a physiologically acceptable medium.

An aspect of the invention is also a care, make-up or treatment cosmetic process for keratin materials of human beings, and in particular the skin, the lips and superficial body s growths, comprising the application to the keratin materials of the composition, in particular the cosmetic composition, as defined above.

An aspect of the invention is also a combination (i) of at least one polymer consisting of a polymer (homopolymer or copolymer) with a weight-average molecular mass ranging from 500 to 500,000, containing at least one moiety comprising:

at least one polyorganosiloxane group, consisting of from 1 to 1,000 organosiloxane units in the chain of the moiety or in the form of a graft, and

at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,

the polymer being solid at room temperature and soluble in said oil at a temperature of from 25 to 250° C. and

(ii) at least one film-forming agent,

in a cosmetic composition or for the manufacture of a physiologically acceptable composition, to obtain a solid composition, such as a wax-free composition, which does not exude and/or which can produce a glossy and/or comfortable deposit on keratin materials, said composition containing an said oil having an affinity with said structuring polymer and optionally with the film-forming agent, and the liquid fatty phase, the polymer and the film-forming agent forming a physiologically acceptable medium.

The compositions of the present invention may also further comprise water, optionally thickened with an aqueous-phase thickener or gelled with a film-forming agent and/or containing ingredients soluble in water.

The invention is illustrated in greater detail in the examples, which follow. The amounts are given as percentages by mass.

### EXAMPLE 1

### Lip Gloss

| Ingredient | Trade Name | % w/w |
|---|---|---|
| Film Former | SA-70 from 3M | 20.0 |
| Polyamidodimethylsiloxane¹ | DC2-8179 | 8.0 |
| Phenyltrimethicone | DC 556 | 65.1 |
| Pigments | | 6.9 |

¹Dow Corning DC 2-9179 (DP = 15)

This composition was supple and elastic.

The film forming polymer is introduced under agitation with a magnetic stirrer after the rest of the formula has been heated. The gloss is introduced into a container and applied using a sponge type applicator.

The composition exhibits better wear when compared with one not containing a film-forming polymer.

### EXAMPLE 2

### Foundation

In phase A, ingredients are mixed well and ground with a Silverson homogenizer at a speed of 6000 rpm.

Separately the phase B1 ingredients are heated to 80 to 85° C. with stirring for 10-15 minutes or until dissolution of the siloxane-polyamide.

Phase A and B1 are then combined in the main beaker and mixed well at 70 to 75° C.

Phase B2 is added to the main beaker and is mixed until uniform.

Disteardimonium Hectorite is added to the main beaker and dispersed well before adding rest of phase B3 ingredients.

Phase C is heated to 70 to 75° C. in a separate side beaker. Emulsification is carried out by adding phase C to main beaker and homogenizing at medium/high speed.

The batch is cooled to room temperature with a paddle stirrer.

| | | |
|---|---|---|
| A | Cyclopentasiloxane (and) dmethicone copolyol | 8.0 |
| | Polyglyceryl-4 isostearate (and) hexyl laurate (and) cetyl | 3.5 |
| | PEG/PPG-10/1 dimethicone | |
| | Treated pigments | 9.9 |
| B1 | Cyclopentasiloxane | 16.1 |
| | Polysiloxane/Polyamide | 1.0 |
| | Silicone-Acrylates | 12.0 |
| B2 | Fillers | 6.0 |
| B3 | Preservative | 0.4 |
| | Disteardimonium Hectorite | 0.6 |
| | Propylene Carbonate | 0.2 |
| C | Water | Qsp 100 |
| | Magnesium Sulfate | 1.0 |
| | Preservative | 0.7 |
| | Non ionic emulsifier | 0.5 |
| | TOTAL | 100.00 |

### EXAMPLE 3

### Foundation

Is prepared according to the same procedure as described in example 2.

| Phase | Ingredient Name | % w/w |
|---|---|---|
| A | Cyclopentasiloxane and Dimethicone Copolyol | 8.00 |
| | Polyglyceryl-4-isostearate and Hexyl Laurate and Cetyl | 3.50 |
| | PEG/PPG-10/1 Dimethicone2 | |
| | Treated Pigments | 9.90 |
| B1 | Volatile Oil | 26.10 |
| | Siloxane based polyamide | 2.00 |
| | TiO₂/Silicone-Acrylates | 12.00 |
| B2 | Fillers | 6.00 |
| B3 | Preservative | 0.40 |
| | Disteardimonium Hectorite | 1.00 |
| | Propylene Carbonate | 0.30 |
| C | Water | qsp |
| | Magnesium Sulfate | 1.00 |
| | Preservatives | 0.70 |
| | Laureth-4 | 0.50 |
| | | 100.00 |

1. Composition comprising at least one liquid fatty phase comprising
(i) at least one oil structured with at least one structuring polymer consisting of a polymer (homopolymer or copolymer) with a weight-average molecular mass ranging from 500 to 500,000, containing at least one moiety comprising:
at least one polyorganosiloxane group, consisting of from 1 to 1 000 organosiloxane units in the chain of the moiety or in the form of a graft, and
at least two groups capable of establishing hydrogen interactions, chosen from ester, amide, sulphonamide, carbamate, thiocarbamate, urea, thiourea, oxamido, guanamido and biguanidino groups, and combinations thereof, on condition that at least one of the groups is other than an ester group,
the polymer being solid at 25° C and soluble in said oil at a temperature of from 25 to 250° C. and
(ii) at least one film-forming polymer,
said oil having an affinity with said structuring polymer and optionally said film-forming polymer, and
the liquid fatty phase, the structuring polymer and the film-forming polymer forming a physiologically acceptable medium.

2. Composition according to claim 1, in which the structuring polymer comprises at least one moiety corresponding to the formula: 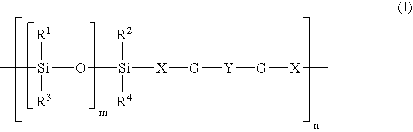
in which:
1) R¹, R², R³ and R⁴, which may be identical or different, represent a group chosen from:
linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulphur and/or nitrogen atoms;
2) the groups X, which may be identical or different, represent a linear or branched C₁ to C₃₀ alkylenediyl group, possibly containing in its chain one or more oxygen and/or nitrogen atoms;
3) Y is a saturated or unsaturated, C₁ to C₅₀ linear or branched divalent alkylene, arylene, cycloalkylene, alkylarylene or arylalkylene group, possibly comprising one or more oxygen, sulphur and/or nitrogen atoms, and/or bearing as substituent one of the following atoms or groups of atoms:
fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or
4) Y represents a group corresponding to the formula: 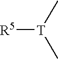
in which
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R⁵ represents a linear or branched C₁ to C₅₀ alkyl group or a polyorganosiloxane chain, possibly comprising one or more ester, amide, urethane, thiocarbamate, urea, thiourea and/or sulphonamide groups, which may possibly be linked to another chain of the polymer;
5) The groups G, which may be identical or different, represent divalent groups chosen from: 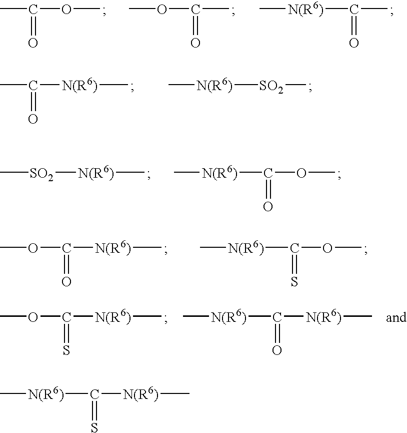
in which R⁶ represents a hydrogen atom or a linear or branched C₁ to C₂₀ alkyl group, on condition that at least 50% of the groups R⁶ of the polymer represents a hydrogen atom and that at least two of the groups G of the polymer are a group other than: 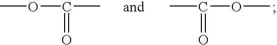
6) n is an integer ranging from 2 to 500 and preferably from 2 to 200, and m is an integer ranging from 1 to 1,000, preferably from 1 to 700 and better still from 6 to 200.

3. Composition according to claim 2, in which Y represents a group chosen from:
a) linear C₁ to C₂₀ and preferably C₁ to C₁₀ alkylene groups,
b) C₃₀ to C₅₆ branched alkylene groups possibly comprising rings and unconjugated unsaturations,
c) C₅-C₆ cycloalkylene groups,
d) phenylene groups optionally substituted with one or more C₁ to C₄₀ alkyl groups,
e) C₁ to C₂₀ alkylene groups comprising from 1 to 5 amide groups,
f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,
g) polyorganosiloxane chains of formula: 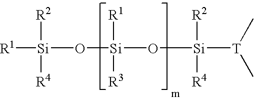
in which R¹, R², R³, R⁴, T and m are as defined above,
h) polyorganosiloxane chains of formula: 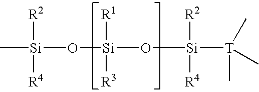
in which R¹, R², R³, R⁴, T and m are as defined above.

4. Composition according to claim 1, in which the structuring polymer comprises at least one moiety corresponding to formula (II): 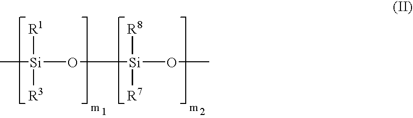
in which
R¹ and R³, which may be identical or different, are as defined above for formula (I) in claim 10,
R⁷ represents a group as defined above for R¹ and R³, or represents a group of formula —X—G—R⁹ in which X and G are as defined above for formula (I) in claim 10, and R⁹ represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,
R⁸ represents a group of formula —X—G—R⁹ in which X, G and R⁹ are as defined above,
m₁ is an integer ranging from 1 to 998, and
m₂ is an integer ranging from 2 to 500.

5. Composition according to claim 3, in which the polymer comprises at least one moiety of formula (III) or (IV): 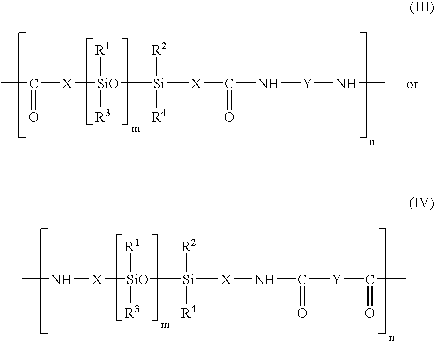
in which R¹, R², R³, R⁴, X, Y, m and n are as defined in claim 10.

6. Composition according to either of claim 2, in which X and/or Y represent an alkylene group containing in its alkylene portion at least one of the following elements:
1°) 1 to 5 amide, urea or carbamate groups,
2°) a C₅ or C₆ cycloalkyl group, and
3°) a phenylene group optionally substituted with 1 to 3 identical or different C₁ to C₃ alkyl groups, and/or substituted with at least one element chosen from the group consisting of:
a hydroxyl group,
a C₃ to C₈ cycloalkyl group,
one to three C₁ to C₄₀ alkyl groups,
a phenyl group optionally substituted with one to three C₁ to C₃ alkyl groups,
a C₁ to C₃ hydroxyalkyl group, and
a C₁ to C₆ aminoalkyl group.

7. Composition according to claim 2, in which Y represents: 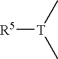
in which R⁵ represents a polyorganosiloxane chain and T represents a group of formula: 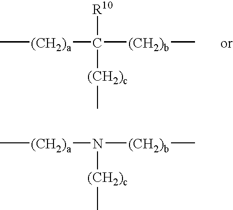
in which a, b and c are, independents, integers ranging from 1 to 10, and R¹⁰ is a hydrogen atom or a group such as those defined for R¹, R², R³ and R⁴, in claim 20.

8. Composition according to claim 2, in which R¹, R², R³ and R⁴ represent, independently, a linear or branched C₁ to C₄₀ alkyl group, preferably a CH₃, C₂H₅, n—C₃H₇ or isopropyl group, a polyorganosiloxane chain or a phenyl group optionally substituted with one to three methyl or ethyl groups.

9. Composition according to claim 1, in which the structuring polymer comprises at least one moiety of formula: 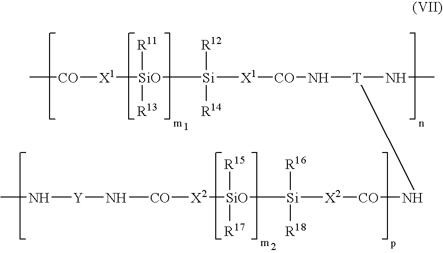
in which X¹ and X², which may be identical or different, have the meaning given for X in claim 10, n, Y and T are as defined in claim 10, R¹¹ to R¹⁸ are groups chosen from the same group as R¹to R⁴ of claim 10, m₁ and m₂ are numbers in the range from 1 to 1 000, and p is an integer ranging from 2 to 500.

10. Composition according to claim 9, in which:
p is in the range from 1 to 25 and better still from 1 to 7,
R¹¹ to R¹⁸ are methyl groups,
T corresponds to one of the following formulae: 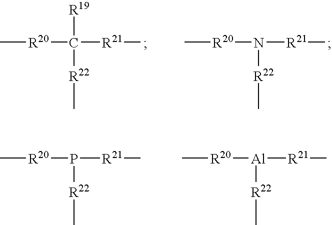
in which R¹⁹ is a hydrogen atom or a group chosen from the groups defined for R¹ to R⁴, and R²⁰, R²¹ and R²² are, independently, linear or branched alkylene groups, and more preferably corresponds to the formula: 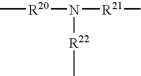
in particular with R²⁰, R²¹ and R²² representing —CH₂—CH₂—,
m₁ and m₂ are in the range from 15 to 500 and better still from 15 to 45,
X¹ and X² represent —(CH₂)₁₀—, and
Y represents —CH₂—.

11. Composition according to claim 1, in which the polymer comprises at least one moiety corresponding to the following formula: 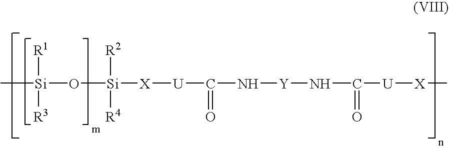
in which R¹, R², R³, R⁴, X, Y, m and n have the meanings given above for formula (I) in claim 20, and U represents —O— or —NH—, such that: 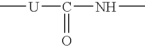
corresponds to a urethane or urea group, or
Y represents a C₅ to C₁₂ cycloaliphatic or aromatic group that may be substituted with a C₁ to C₁₅ alkyl group or a C₅ to C₁₀ aryl group, for example a radical chosen from the methylene-4,4-biscyclohexyl radical, the radical derived from isophorone diisocyanate, 2,4-and 2,6-tolylenes, 1,5-naphthylene, p-phenylene and 4,4′-biphenylenemethane or
Y represents a linear or branched C₁ to C₄₀ alkylene radical or a C₄ to C₁₂ cycloalkylene radical, or
Y represents a polyurethane or polyurea block corresponding to the condensation of several diisocyanate molecules with one or more coupling agents of the diol or diamine type, corresponding to the formula: 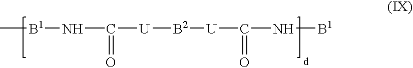
in which B¹ is a group chosen from the groups given above for Y, U is —O— or —NH— and B² is chosen from:
linear or branched C₁ to C₄₀ alkylene groups, which can optionally bear an ionizable group such as a carboxylic acid or sulphonic acid group, or a neutralizable or quaternizable tertiary amine group,
C₅ to C₁₂ cycloalkylene groups, optionally bearing alkyl substituents, for example one to three methyl or ethyl groups, or alkylene, for example the diol radical: cyclohexanedimethanol,
phenylene groups that may optionally bear C₁ to C₃ alkyl substituents, and
groups of formula: 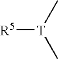
in which T is a hydrocarbon-based trivalent radical possibly containing one or more hetero atoms such as oxygen, sulphur and nitrogen and R⁵ is a polyorganosiloxane chain or a linear or branched C₁ to C₅₀ alkyl chain.

12. Composition according to claim 1, in which the polymer comprises at least one moiety of formula: 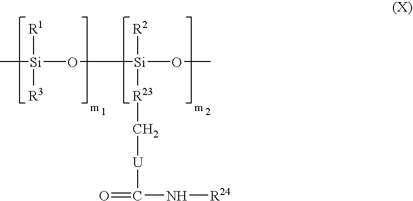
in which R¹, R², R³, m₁ and m₂ have the meanings given above for formula (I),
U represents O or NH,
R²³ represents a C₁ to C₄₀ alkylene group, optionally comprising one or more hetero atoms chosen from O and N, or a phenylene group, and
R²⁴ is chosen from linear, branched or cyclic, saturated or unsaturated C₁ to C50 alkyl groups, and phenyl groups optionally substituted with one to three C₁ to C₃ alkyl groups.

13. Composition according to claim 1, in which the structuring polymer comprises at least one moiety of formula: 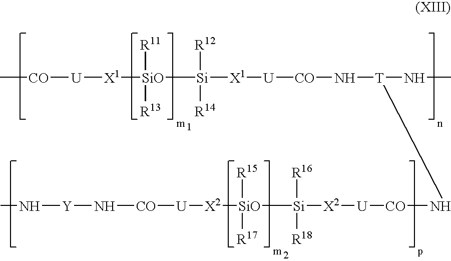
in which X¹ and X², which are identical or different, have the meaning given for X in claim 10, n, Y and T are as defined in claim 10, R¹¹ to R¹⁸ are groups chosen from the same group as R¹ to R⁴ of claim 10, m₁ and m₂ are numbers in the range from 1 to 1 000, and p is an integer ranging from 2 to 500.

14. Composition according to claim 2, in which the structuring polymer furthermore comprises a hydrocarbon-based moiety comprising two groups capable of establishing hydrogen interactions, chosen from ester, amide, sulphonamide, carbamate, thiocarbamate, urea, thiourea, oxamido, guanamido and biguanidino groups, and combinations thereof.

15. Composition according to claim 14, in which the copolymer is a block copolymer or a grafted copolymer.

16. Composition according to claim 1, in which the polymer represents from 0.5% to 80%, preferably from 2% to 60% and better still from 5% to 40%, relative to the total weight of the composition.

17. Composition according to claim 1, wherein said at least one structuring polymer has a softening point greater than 50° C.

18. Composition according to claim 1, wherein said at least one structuring polymer has a softening point is less than 150° C.

19. Composition according to claim 1, wherein said at least one structuring polymer has a softening point ranging from 70° C. to 130° C.

20. Composition according to claim 1, wherein said at least one structuring polymer has a weight-average molecular mass ranging from 500 to 200,000, preferably ranging from 1,000 to 100,000, more preferably ranging from 2,000 to 30,000.

21. Composition according to claim 1, wherein said composition has a hardness ranging from 30 to 300 gf.

22. Composition according to claim 1, wherein said composition has a hardness ranging from 30 to 250 gf, preferably from 30 to 200 gf.

23. Composition according to claim 1, wherein said at least one liquid fatty phase of the composition comprises at least one oil chosen from at least one polar oil and at least one apolar oil having an affinity with the least one structuring polymer.

24. Composition according to claim 23, wherein said at least one polar oil is chosen from:
hydrocarbon-based plant oils with a high content of triglycerides comprising fatty acid esters of glycerol in which the fatty acids comprise chains having from 4 to 24 carbon atoms, said chains optionally being chosen from linear and branched, and saturated and unsaturated chains;
synthetic oils or esters of formula R₅COOR₆ in which R₅ is chosen from linear and branched fatty acid residues comprising from 1 to 40 carbon atoms and R₆ is chosen from hydrocarbon-based chain containing form 1 to 40 carbon atoms, with the proviso that R₅+R₆≧10;
synthetic ethers containing from 10 to 40 carbon atoms;
C₈ to C₂₆ fatty alcohols; and
C₈ to C₂₆ fatty acids.

25. Composition according to claim 23, wherein said at least one apolar oil is chosen from:
silicone oils chosen from volatile and non-volatile, linear and cyclic polydimethylsiloxanes that are liquid at room temperature;
polydimethylsiloxanes comprising alkyl or alkoxy groups which are pendant and/or at the end of the silicone chain, the groups each containing from 2 to 24 carbon atoms;
phenylsilicones, in particular phenyltrimethicone; and
hydrocarbons chosen from linear and branched, volatile and non-volatile hydrocarbons of synthetic and mineral origin.

26. Composition according to claim 1, wherein said at least one liquid fatty phase is present in an amount ranging from 1% to 99% by weight relative to the total weight of the composition.

27. Composition according to claim 1, wherein said at least one liquid fatty phase is present in an amount ranging from 10% to 80% by weight relative to the total weight of the composition.

28. Composition according to claim 1, in which the liquid fatty phase contains more than 30%, preferably more than 40% and better still from 50% to 100% by weight of at least one silicone-based liquid oil.

29. Composition according to claim 1, wherein said at least one liquid fatty phase comprises at least one volatile solvent chosen from hydrocarbon-based solvents and silicone solvents optionally comprising alkyl or alkoxy groups that are pendant or at the end of a silicone chain.

30. Composition according to claim 1, wherein said at least one film-forming polymer is chosen from the group comprising:
liposoluble film-forming polymers,
lipodispersible film-forming polymers in the form of non-aqueous dispersions of polymer particles, preferably dispersions in silicone or hydrocarbon oils; in one embodiment, the non-aqueous dispersions of polymer comprise polymer particles stabilised on their surface by at least one stabilising agent; these non-aqueous dispersions are often called “NAD”,
aqueous dispersions of polymer particles, often called “latex”,
water-soluble film-forming polymers.

31. Composition according to claim 30, wherein said at least one film-forming polymer is a film-forming liposoluble polymer.

32. Composition according to claim 31, wherein said at least one film-forming liposoluble polymer is chosen from the liposoluble amorphous homopolymers and copolymers of olefins, cyclo-olefins, butadiene, isoprene, styrene, ethers, vinyl esters or amides, esters or amides of (meth)acrylic acid containing a linear, branched or cyclic C4-C50 alkyl group, and preferably amorphous.

33. Composition according to claim 31, wherein said at least one film-forming liposoluble polymer is chosen from the homopolymers and copolymers obtained from monomers chosen from within the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tertio-butyl (meth)acrylate, tridecyl (meth)acrylate, stearyl (meth)acrylate or mixtures thereof, in particular an alkyl acrylate/cycloalkyl acrylate copolymer and the vinyl pyrrolidone-decadecene copolymers.

34. Composition according to claim 31, wherein said at least one film-forming liposoluble polymer is chosen from the liposoluble amorphous polycondensates, preferably not comprising hydrogen-interaction donor groups, in particular polyesters with C₄₋₅₀ alkyl side chains or else polyesters resulting from the condensation of fatty acid dimers or even polyesters comprising a silicone segment in the form of a sequence, graft or end group, solid at room temperature ° C.

35. Composition according to claim 31, wherein said at least one film-forming liposoluble polymer is chosen from the liposoluble and amorphous polysaccharides comprising alkyl (ether or ester) side chains, in particular ethylcellulose, acrylic-silicone grafted polymers with a silicone backbone, acrylic grafts or with an acrylic backbone, silicone grafts.

36. Composition according to claim 31, wherein said at least one film-forming liposoluble polymer bears fluorous groups.

37. Composition according to claim 36, wherein said at least one film-forming liposoluble polymer is chosen from among the alkyl (meth)acrylate/perfluoroalkyl (meth)acrylate copolymers.

38. Composition according to claim 31, wherein said at least one film-forming liposoluble polymer is chosen from polymers or copolymers resulting from the polymerization or copolymerization of an ethylenic monomer, comprising one or more ethylenic, preferably conjugated bonds (or dienes).

39. Composition according to claim 38, wherein said polymer or copolymer resulting from the polymerization or copolymerization of an ethylenic monomer, comprising one or more ethylenic is of the polystyrene/copoly(ethylene/butylene) type.

40. Composition according to claim 31, wherein said at least one film-forming liposoluble polymer has a molecular weight ranging between 1,000 and 500,000, preferably between 2,000 and 250,000, and a glass transition temperature ranging between −100° C. and +300° C. preferably between −50° C. and +100° C. preferably still between −10° C. and +90° C.

41. Composition according to claim 31, wherein said at least one film-forming liposoluble polymer is chosen from the polymers with a non-silicone organic backbone grafted with monomers containing a polysiloxane.

42. Composition according to claim 41, wherein the polymers with a non-silicone organic backbone grafted with monomers containing a polysiloxane are grafted silicone copolymers containing:
a) from 0 to 98% by weight of at least one lipophilic monomer (A) of low lipophilic polarity with ethylene unsaturation, polymerizable by the radical method;
b) from 0 to 98% by weight of at least one hydrophilic polar monomer (B) with ethylene unsaturation, copolymerizable with the monomer or monomers of type (A);
c) from 0.01 to 50% by weight of at least one polysiloxane macromer (C) of general formula:
X(Y)ₙSi(R)₃₋ₘZₘ (I)
in which:
X designates a vinyl group copolymerizable with the monomers (A) and (B);
Y designates a group with a divalent bond;
R designates a hydrogen, an C₁-C₆ alkyl or alkoxy, a C₆-C₁₂ aryl;
Z designates a monovalent polysiloxane moiety having an average molecular weight by number of at least 500;
n is 0 or 1 and m is an integer ranging from 1 to 3.
n being a number ranging from 5 to 700 and I being an integer ranging between 0 and 3 [sic].

43. Composition according to claim 31, wherein said at least one film-forming liposoluble polymer is chosen from the silicone polymers grafted with non-silicone organic monomers.

44. Composition according to claim 43, wherein said grafted silicone polymer containing a main silicone chain is composed of the result of radical copolymerization between, on the one hand, at least one non-silicone anionic organic monomer having an ethylene unsaturation and/or a non-silicone hydrophobic organic monomer having an ethylene unsaturation and, on the other, a silicone having in its chain at least one, preferably several, functional groups capable of reacting on said ethylene unsaturations of said non-silicone monomers by forming a covalent bond, in particular thio-functional groups.

45. Composition according to claim 44, wherein said anionic monomers with ethylene unsaturation are chosen, alone or as a mixture, from among the linear or branched unsaturated carboxylic acids, possibly neutralized in whole or in part in the form of a salt, this or these unsaturated carboxylic acid(s) being able to be most particularly acrylic acid, methacrylic acid, maleic acid, maleic anhydride, itaconic acid, fumaric acid and crotonic acid.

46. Composition according to claim 44, wherein the hydrophobic monomers with ethylene unsaturation preferably are chosen from among the alkanol acrylic acid esters and/or the alkanol methacrylic acid esters. The alkanols preferably are C₁-C₁₈ and more particularly C₁-C₁₂. The preferred monomers are chosen from within the group consisting of isooctyl (meth)acrylate, isononyl (meth)acrylate, 2-ethylhexyl (meth)acrylate, lauryl (meth)acrylate, isopentyi (meth)acrylate, n-butyl (meth)acrylate, isobutyl (meth)acrylate, methyl (meth)acrylate, tertio-butyl (meth) acrylate, tridecyl (meth)acrylate, stearyl (meth)acrylate or mixtures thereof.

47. Composition according to claim 30, wherein said at least one film-forming polymer is chosen from the lipodispersible film-forming polymers in the form of non-aqueous dispersions of polymer particles, preferably dispersions in silicone or hydrocarbon oils.

48. Composition according to claim 30, wherein said at least one film-forming polymer is water-dispersible.

49. Composition according to claim 48, wherein said at least one film-forming water-dispersible polymer is chosen from polymers with a non-silicone organic backbone grafted with monomers containing a polysiloxane.

50. Composition according to claim 49, wherein the polymers with a non-silicone organic backbone grafted with monomers containing a polysiloxane are grafted silicone copolymers containing:
a) from 0 to 98% by weight of at least one lipophilic monomer (A) of low lipophilic polarity with ethylene unsaturation, polymerizable by the radical method;
b) from 0 to 98% by weight of at least one hydrophilic polar monomer (B) with ethylene unsaturation, copolymerizable with the monomer or monomers of type (A);
c) from 0.01 to 50% by weight of at least one polysiloxane macromer (C) of general formula:
X(Y)ₙSi(R)₃₋ₘZₘ (I)
in which:
X designates a vinyl group copolymerizable with the monomers (A) and (B);
Y designates a group with a divalent bond;
R designates a hydrogen, a C₁-C₆ alkyl or alkoxy, a C₆-C₁₂ aryl;
Z designates a monovalent polysiloxane moiety having an average molecular weight by number of at least 500;
n is 0 or 1 and m is an integer ranging from 1 to 3.
n being a number ranging from 5 to 700 and I being an integer ranging between 0 and 3 [sic].

51. Composition according to claim 48, wherein said at least one film-forming water-dispersible polymer is chosen from the silicone polymers grafted with non-silicone organic monomers.

52. Composition according to claim 51, wherein said grafted silicone polymer containing a main silicone chain is composed of the result of radical copolymerization between, on the one hand, at least one non-silicone anionic organic monomer having an ethylene unsaturation and/or a non-silicone hydrophobic organic monomer having an ethylene unsaturation and, on the other, a silicone having in its chain at least one, and preferably several, functional groups capable of reacting on said ethylene unsaturations of said non-silicone monomers by forming a covalent bond, in particular thio-functional groups.

53. Composition according to claim 52, wherein said anionic monomers with ethylene unsaturation are chosen, alone or as a mixture, from among the linear or branched unsaturated carboxylic acids, possibly neutralized in whole or in part in the form of a salt, this or these unsaturated carboxylic acid(s) being able to be most particularly acrylic acid, methacrylic acid, maleic acid, maleic anhydride, itaconic acid, fumaric acid and crotonic acid.

54. Composition according to claim 52, wherein the hydrophobic monomers with ethylene unsaturation preferably are chosen from among the alkanol esters of acrylic acid and/or the alkanol esters of methacrylic acid. The alkanols preferably are C₁-C₁₈ and more particularly C₁-C₁₂. The preferred monomers are chosen from within the group consisting of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tertio-butyl (meth)acrylate, tridecyl (meth)acrylate, stearyl (meth)acrylate or mixtures thereof.

55. Composition according to claim 1, wherein the film-forming weight ratio ranges from 2-60%, preferably from 5 to 60%, more preferably from 2 to 30% by weight of dry compound relative to the total weight of the composition.

56. Composition according to claim 1, further comprising at least one additional additive chosen from dispersants such as poly(2-hydroxystearic acid), antioxidants, essential oils, preserving agents, fragrances, waxes, fillers, neutralizing agents, cosmetic and dermatological active agents such as, for example, emollients, moisturizers, vitamins, essential fatty acids, sunscreens, and mixtures thereof.

57. Composition according to claim 1, further comprising at least one coloring agent

58. Composition according to claim 1, wherein said at least one coloring agent is chosen from lipophilic dyes, hydrophilic dyes, pigments and nacres.

59. Composition according to claim 57, wherein said at least one coloring agent is present in a proportion of from 0.01% to 50%, preferably from 0.5% to 40% and more preferably from 5% to 30% relative to the total weight of the composition.

60. Composition according to claim 1, wherein said composition is a solid.

61. Composition according to any one of the preceding claims, wherein said composition is a solid chosen from molded and poured sticks.

62. Composition according to claim 1, wherein said composition is in the form of a rigid gel.

63. Composition according to claim 1, wherein said composition is in the form of an anhydrous stick.

64. Composition according to claim 1, wherein said composition contains an aqueous phase.

65. Composition according to claim 1, wherein said composition is an emulsion.

66. A make-up or care or treatment composition for the skin, the lips, or keratinous fibers containing at least one liquid fatty phase comprising (i) at least one oil structured with at least one structuring polymer as described in claim 1,
and (ii) at least one film-forming polymer,
said oil having an affinity with said structuring polymer and optionally with the film-forming polymer, and
the liquid fatty phase, the polymer and the film-forming polymer forming a physiologically acceptable medium.

67. A make-up or care or treatment composition for the skin, the lips, or keratinous fibers comprising at least one liquid fatty phase comprising (i) at least one oil structured with at least one structuring polymer as described in claim 1,
(ii) at least one film-forming polymer, and at least one coloring agent, said oil having an affinity with said structuring polymer and optionally with the film-forming polymer, and the liquid fatty phase, the polymer and the film-forming polymer forming a physiologically acceptable medium.

68. A mascara, an eyeliner, a foundation, a lipstick, a blusher, a make-up-removing product, a make-up product for the body, an eyeshadow, a face powder, a concealer product, a shampoo, a conditioner, an antisun product or a care product for the lips, skin, or hair comprising a composition comprising at least one liquid fatty phase in the mascara, eyeliner, foundation, lipstick, blusher, make-up-removing product, make-up product for the body, eyeshadow, face powder, concealer product, shampoo, conditioner, antisun product or care product for the skin, lips, or hair which comprises at least one liquid fatty phase comprising (i) at least one oil structured with at least one structuring polymer as described in claim 1,
and (ii) at least one film-forming polymer,
said oil having an affinity with said structuring polymer and optionally with the film-forming polymer, and
the liquid fatty phase, the polymer and the film-forming polymer forming a physiologically acceptable medium.

69. A care product for the skin or body comprising an anhydrous composition containing at least one liquid fatty phase comprising (i) at least one oil structured with at least one structuring polymer as described in claim 1,
and (ii) at least one film-forming polymer,
said oil having an affinity with said structuring polymer and optionally with the film-forming polymer, and
the liquid fatty phase, the polymer and the film-forming polymer forming a physiologically acceptable medium.

70. A lipstick composition comprising an anhydrous composition containing at least one liquid fatty phase comprising (i) at least one oil structured with at least one structuring polymer as described in claim 1,
and (ii) at least one film-forming polymer,
said oil having an affinity with said structuring polymer and optionally with the film-forming polymer, and
the liquid fatty phase, the polymer and the film-forming polymer forming a physiologically acceptable medium.

71. A method for care, make-up or treatment of keratin materials comprising applying to the keratin materials an anhydrous composition containing at least one liquid fatty phase comprising (i) at least one oil structured with at least one structuring polymer as described in claim 1,
and (ii) at least one film-forming polymer,
said oil having an affinity with said structuring polymer and optionally with the film-forming polymer, and
the liquid fatty phase, the polymer and the film-forming polymer forming a physiologically acceptable medium.

72. A method for care, make-up or treatment of keratinous fibers, lips, or skin comprising applying to the keratinous fibers, lips, or skin a composition comprising at least one liquid fatty phase comprising (i) at least one oil structured with at least one structuring polymer described in claim 1,
and (ii) at least one film-forming polymer,
said oil having an affinity with said structuring polymer and optionally with the film-forming polymer, and
the liquid fatty phase, the polymer and the film-forming polymer forming a physiologically acceptable medium.

73. A method for providing an anhydrous composition having at least one property chosen from non-exudation, gloss, and comfortable deposit on keratin materials chosen from lips, skin, and keratinous fibers, comprising including in the composition at least one liquid fatty phase comprising (i) at least one oil structured with at least one structuring polymer as described in claim 1,
and (ii) at least one film-forming polymer,
said oil having an affinity with said structuring polymer and optionally with the film-forming polymer, and
the liquid fatty phase, the polymer and the film-forming polymer forming a physiologically acceptable medium.

74. A method of making up or caring for skin, lips or keratinous fibers comprising applying to the skin, lips, or keratinous fibers a structured composition containing at least one liquid fatty phase comprising (i) at least one oil structured with at least one structuring polymer as described in claim 1,
and (ii) at least one film-forming polymer,
said oil having an affinity with said structuring polymer and optionally with the film-forming polymer, and the liquid fatty phase, the polymer and the film-forming polymer forming a physiologically acceptable medium.
